(19) United States
(12) Patent Application Publication
(10) Pub. No.: US 2004/0006036 A1
(43) Pub. Date: Jan. 8, 2004

(54) Silencing transcription by methylation

(75) Inventors: Ji-Fan Hu, Palo Alto, CA (US); Scott Bowersox, Menlo Park, CA (US)

Correspondence Address: QUARLES & BRADY LLP, RENAISSANCE ONE, TWO NORTH CENTRAL AVENUE, PHOENIX, AZ, 85004-2391, US

(73) Assignee: GMR, a Delaware corporation

(21) Appl. No.: 10/422,466

(22) Filed: Apr. 22, 2003

Related U.S. Application Data
(63) Continuation-in-part of application No. 09/643,128, filed on Aug. 21, 2000.
(60) Provisional application No. 60/196,749, filed on Apr. 12, 2000.
(60) Provisional application No. 60/214,148, filed on Jun. 26, 2000.

(51) Int. Cl. A61K 48/00; C07H 21/04

(52) U.S. Cl. 514/44; 536/23.1

(57) ABSTRACT

The invention provides methods and compositions related to oligonucleotides that silence target genes within a cell. The oligonucleotides include an oligonucleotide methylator segment that has a first strand and a second strand complementary to the first strand. The first strand can include at least one m5CG sequence which is paired with an unmethylated CG sequence on the second strand. Alternatively, the first strand can include at least one m5CNIG sequence paired with an unmethylated CN2G sequence on the second strand, wherein N1 is any nucleotide, and N2 is a nucleotide that pairs with N1. The oligonucleotides also include a single-stranded DNA binding segment that is complementary to a nucleotide sequence in the target gene. The DNA binding segment includes at least one m5CG sequence m5CG or at least one 5CN3G sequence, wherein N3 is any nucleotide. The methylator segment and DNA binding segment are operably linked such that the oligonucleotide is capable of inducing methylation at the target nucleotide sequence, thereby silencing the target gene.

### CROSS REFERENCE TO RELATED APPLICATIONS

This is a continuationin part of U.S. Ser. No. 09/643,128, filed on Aug. 21, 2000, which claims the benefit of provisional application No. 60/196,749, filed Apr. 12, 2000, and provisional application No. 60/214,148, filed Jun. 26, 2000, the entire disclosures of which are incorporated herein by reference.

### STATEMENT REGARDING FEDERALLY SPONSORED RESEARCH

This work was funded in part by a Merit Review Grant from the Department of Veterans Affairs. The U.S. government may have certain rights in this invention.

### BACKGROUND

1. Technical Field

The present invention relates to novel drug compounds and methods to treat medical conditions associated with excessive or abnormal expression of proteins, more particularly to alter transcription of oncogenes and other genes.

2. The Prior Art

Gene expression can be regulated by methylation. Many genes are known to be inactivated by hypermethylation. Such genes are not transcribed and are the to be “silenced.” DNA methylation is also a common epigenetic mechanism involved in the regulation of so-called “imprinted” genes, which are uni-parentally or mono-allelically expressed. For example, the Igf2 gene is normally imprinted in tissues, with only the paternal allele being transcribed (De Chiara, T. M. et al. (1990) Nature 345:78-80). Expression of the human Igf2 is controlled by four upstream promoters (hP1-hP4). The first promoter (hP1) is located just next to the insulin gene and is not subject to genomic imprinting. The remaining three promoters (hP2-hP4) are all monoallelically expressed from the paternal allele (Vu, T. H. and Hoffman, A. (1994) Nature 371:714-717). It was thus proposed that an imprinting maintenance element (IME) is located between the non-imprinted hP1 and the three hP2-hP4 promoters and controls the allelic expression of Igf2.

Because methylation controls gene expression over a long time period, methods for directed DNA methylation have been sought. Holliday (U.S. Pat. No. 5,840,497) discloses a method for silencing genes that involves the use of a single stranded oligonucleotide containing 5-methyl deoxycytidine residues, wherein the oligonucleotide is complementary to a gene of interest. Binding of the methylated oligonucleotide to a complementary sequence induces methylation of the complementary sequence by a nuclear DNA methylase. However, this method is not efficient enough to allow control of gene expression in vivo. Accordingly, there remains a need for efficient means of controlling gene expression in cells by targeted methylation. Hu (WO01/79441 A2) discloses an efficient method of silencing a gene using a special IE/GE compound. This IE/GE oligonucleotide compound had a guiding element (GE) complementary to the gene sequence. An “inactivating element” (IE) comprised an 11-mer oligonucleotide duplex. IE/GE compounds were highly effective in inducing DNA methylation and thus silencing the target genes. However, this long IE/GE oligo is expensive to make and is long enough to form unwanted secondary structures, which can interfere with the compound's ability to bind nucleic acid specifically and to form the imprinting duplex. The size of IE/GE constructs may also affect their biodistribution, i.e., ability to penetrate cells and move to their sites of action (genomic DNA). It would be preferable to increase the length of the guiding element and increase specificity of action while decreasing side effects. What is needed is a more specific compound that has more desirable physicochemical properties for pharmaceutical use, is less costly to make and more efficiently induces gene silencing.

### BRIEF SUMMARY OF THE INVENTION

It is one object of this invention to provide oligonucleotides that specifically target genes that are abnormally turned on during cancer states.

The present invention is a method of creating chemical compounds that act on nuclear DNA to silence gene transcription by inducing methylation of a nucleotide target sequence. DNA methylation is a powerful, endogenous molecular mechanism by which cells silence both endogenous and exogenous genes.

The object of this invention is to silence genes by administering a modified oligonucleotide that contains a segment (“DNA binding segment”) that is complementary to either the template (non-sense) or the sense strand of a specific genomic DNA sequence and a methylator segment that forms a short, self-annealed hairpin structure at the binding segment's 5′-end or 3′-end. The hairpin structure is a homoduplex composed of a oligonucleotide strand and its complementary base strand. A loop composed of one or more nucleotides forms the stem of the hairpin. The stem contains one or more 5′-CG-3′ dinucleotides or 5′-CNG-3′ trinucleotides, in which “N” is any mucleotide and the cytosine (C) residue is replaced by 5′-methyl cytosine (m5C) in one strand while the other strand has the unmethylated cytosine (C). After self-annealing, it will form a semi-methylated, CG containing, hairpin structure via the loop linker between two strands.

The single stranded portion of the construct (the DNA binding segment) contains one or more CG dinucleotides or CNG trinucleotides, in which the cytosine (C) residue is replaced by 5′-methyl cytosine (m5C). The single strand targeting element is complementary to either the template or coding strand of the target gene sequence. It functions to guide the compound to a specific DNA site. After associating with the target nucleotide sequence, the construct forms a semi-methylated, replication fork-like structure, which is believed to activate the endogenous DNA methylation maintenance enzyme (Dnmt1). During DNA replication, one of the key functions of Dnmt1 is to coordinate with DNA replication machinery and to add a methyl group at the 5′-position of cytosine in the newly synthesized DNA strand. As a result, a full DNA methylation pattern is maintained in daughter DNA.

Compounds of this invention are believed to recruit Dnmt1, which uses the semi-methylated, replication fork-like structure as the substrate and transfers the methyl group to a CG site at the target sequence. Due to a mechanism of DNA spreading, the target sequence may become fully methylated and transcriptionally silenced.

The hairpin structure and the single stranded targeting element are operably linked by an oligonucleotide synthesizer during oligo synthesis or by other chemical methods. The hairpin structure can be placed at either the 5′-end or the 3′-end of the oligonucleotide compound. For example, a preferred silencing compound that targets the most proximal promoter of the human Igf2 has the sequence of CAGCC^{m}5CGGGCTGGGAGGAGT^{m}5CG. The 5′-end oligo (CAGCC^{m}5C) forms a hemi-methylated hairpin structure with the downstream oligo (GGGCTG). In a Bcl-2 silencing compound (G^{m}5CGTCGCGGCGGTAGCGG^{m}5CG), the 5′-end G^{m}5CG anneals with the downstream CGC through a single loop linker (T) and forms a semi-methylated hairpin structure to induce DNA methylation and thus silencing the human Bcl-2 gene.

The invention also provides a method for targeted DNA silencing wherein introduction of a oligonucleotide into a cell is believed to induce methylation at a target nucleotide sequence in the cell. The method can be used with any methylation-competent cell and is preferably applied to mammalian cells (especially human cells), plant cells, or prokaryotic cells. The method encompasses the introduction of oligonucleotides of in vivo as well as ex vivo.

In one embodiment, useful in research, the target nucleotide sequence is in a gene encoding a protein of unknown function. In this case, the method typically includes determining a phenotypic change associated with silencing at the target gene after introduction of the oligonucleotide.

In one embodiment, an organism is produced from a cell after introduction of the oligonucleotide. Typically, in this case, the target sequence is in a gene or gene regulatory region, and the organism either does not express the gene or expresses the gene at a reduced level compared to a normal organism.

In one embodiment of the method, the target nucleotide sequence is a disease causing gene, such as a cancer gene, and methylation at the target nucleotide sequence helps prevent or treat the disease.

In one embodiment of the method, the target nucleotide sequence is an unknown disease gene and methylation at the target nucleotide sequence is applied to gene target validation and gene functional analysis.

In one embodiment of the method, the target nucleotide sequence consist of a transcription suppressor site associated with a target gene, such that methylation at the target nucleotide sequence results in increased expression of the associated gene.

### BRIEF DESCRIPTION OF THE FIGURES

FIG. 1 schematically illustrates the composition and putative mechanism of silencing compounds. The 5′-end of the silencing compound is a self-annealed, hemi-methylated hairpin structure, which is linked by a short loop composed of one or more nucleotides. mC=5′-methyl cytosine. The 3′-end of the silencing compound is a single-stranded oligonucleotide (DNA binding segment), which is complementary to the target sequence in the promoter, enhancer, exons, introns, splicing sites, 3′- or 5′-untranslated regions, suppressors, silencers, and other gene regulatory sequences. The 3′-end of the compound specifically binds to the gene target. After binding to the target sequence, the silencing compound forms a semi-methylated hairpin complex in the local chromatin foci. This structure mimics the DNA replication fork structure formed during DNA replication and is a strong activator of the activity of DNA methyltransferase 1 (Dnmt1). Dnmt1 adds a methyl group at the 5′-position of cytosine of CpG dinucleotide in the target sequence as it usually does at the replication fork site. DNA methylation spreads, so that the whole DNA region is hypermethylated and the target gene becomes silenced.

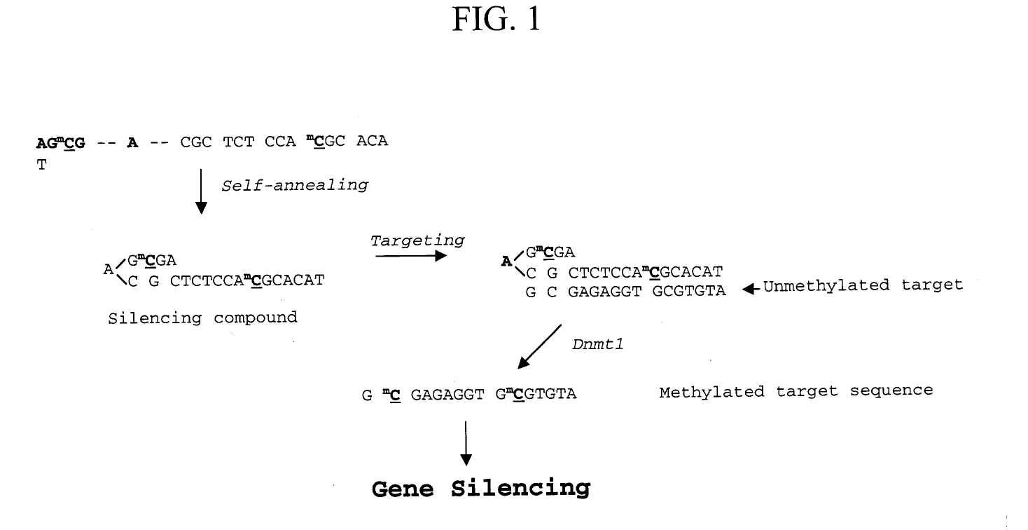

FIG. 2 shows the structure of two test compounds and the inhibition of Igf2 expression by silencing compounds. Hep 3B tumor cells were treated with methylated 22-mer (Hep22M) and a truncated methylated 19-mer (Hep19M). Controls were incubated with phosphate buffered saline (PBS). After 48 hr incubation, total polynucleic acid (TNA) was extracted and was converted into cDNA with reverse transcriptase. The abundance of Igf2 mRNA was quantitated by RT-PCR in duplicated samples. The PCR primers covered the intron and amplified Igf2 DNA and mRNA at the same time. Hep19M is a truncated form of Hep22M lacking the hairpin structure and failing to inhibit Igf2 expression. Thus, the self-annealing, semi-methylated hairpin structure (methylator) is required for maximal Igf2 inhibition.

FIG. 3 shows that some Igf2 silencing compounds protect against tumor death in nude mice. Athymic nude mice were implanted with Hep 3B tumor cells (10⁷ cells). Four weeks after implantation, animals were randomized to receive Hep19M (10 mg/kg, n=13) or Hep22M (10 mg/kg, n=13). All treatments were administered via tail vein injection, twice per week. The Mantel-Cox log rank test showed a significantly prolonged survival in animals receiving Hep22M treatment than those receiving Hep19M treatment (p<0.05). Hep19M, showed no inhibition of Igf2 and no protection against tumor death.

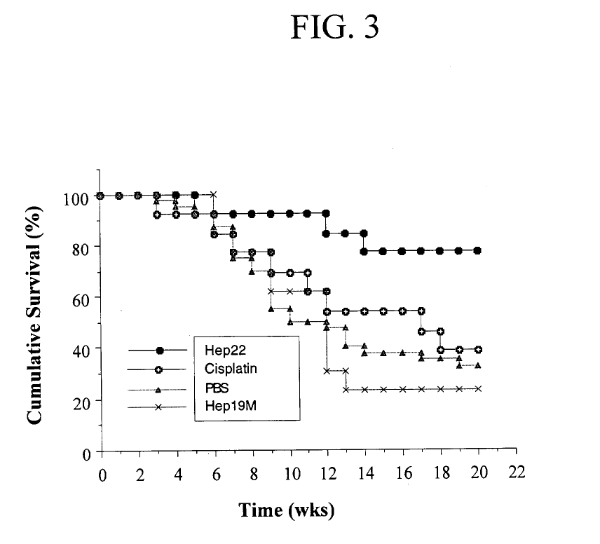

FIG. 4 shows the design of silencing compounds for the Bcl-2 oncogene. Bcl-T1 lacks a semi-methylated hairpin structure and produces much less inhibition than Bclkex-1, -2, and -3 compounds that have the hairpin structure.

FIG. 5 shows the inhibition of Bcl-2 expression by silencing compounds. MCF-7 breast cancer cells were seeded in 24-well plates and treated with test articles (FIG. 4) for 48 hours. Oligos were encapsulated in liposomes before application. Total RNA was extracted and analyzed for Bcl-2 expression by PCR. Bcl-T1 does not contain the hairpin structure and did not block Bcl-2 expression at 10 nM level. Thus, this confirms that the semi-methylated hairpin structure is required for the maximal activity of silencing compounds.

FIG. 6 shows the silencing compound sequences that are designed to silence Bcl-2 gene. These compounds are designed to target the CpG island sequence. They all contain self-annealed, semi-methylated hairpin structures, which induce DNA methylation at the target sequence by harnessing the endogenous gene regulation system.

FIG. 7 shows the effect on the Bcl-2 gene of silencing compounds. Breast cancer (MCF-7) and lung cancer H23 cells were seeded in 6-well plates and were treated for 72 hours with various BclKex compounds (1 μM) encapsulated in liposomes. After incubation, total RNA was extracted for expression analysis of Bcl-2 by PCR amplification.

FIG. 8 shows a western blot of Bcl-2 protein in MCF-7 breast cancels treated with inventive compounds, Genasense and negative controls.

FIG. 9 shows the silencing compound sequences designed to silence TNFα. These compounds contain a self-annealed, semi-methylated hairpin structure, which induces DNA methylation at the target sequence of TNFα by harnessing the endogenous gene regulation system.

FIG. 10 shows the inhibition of TNFα expression by silencing compounds. Lung cancer cells T47D were seeded in 24-well plates and treated with test compounds (0.25 μM) for 48 hours. The compounds were encapsulated in liposomes before application. Total RNA was extracted and analyzed for TNFα expression by PCR. TNFα expression was normalized against the internal control β-actin mRNA and expressed as relative inhibition over the phosphate buffered saline (PBS) control.

FIG. 11 is a bar graph showing the effect of treatment on MKP-1 gene transcription of mRNA with MK-2, MK-3 and MK-9 compared to the liposome carrier control (100%).

FIGS. 12A and 12B show the effect of treatment of MCF-7 cells' expression of MPK-1 protein by Western blot assay (FIG. 12A), with the intensity of the blots analyzed by optical densitometry and reported as a percentage of the liposome carrier control in a bar graph (FIG. 12B).

FIGS. 13A and 13B show the effect of the MK oligos on T47D breast cancer cells. FIG. 13A shows the Western blot assay results, which were analyzed by optical densitometry and reported as a percentage of the Oligofectamine control (FIG. 13B).

FIG. 14 is a bar graph summarizing the effect of CDC25 oligos on CDC25A mRNA production compared to GTS liposomal carrier.

FIGS. 15A and 15B show the effect of CD25 oligos on T47D breast cancer cells. FIG. 15A shows the Western blot assay results, which were analyzed by optical densitometry and reported as a percentage of the Oligofectamine control (FIG. 15B).

### DETAILED DESCRIPTION OF THE INVENTION

The following description of the preferred embodiments of the invention is not intended to limit the scope of the invention to these preferred embodiments, but rather to enable any person skilled in the art of molecular biology to make and use the invention. The invention is based on the discovery that a gene is silenced by targeted DNA methylation using a modified silencing compound that is composed of a semi-methylated hairpin and a single stranded oligonucleotide (DNA binding segment), which is complementary to a particular gene or gene regulatory region.

Definitions

The term “polynucleotide” as used herein refers to any heteropolymer of nucleotide monomers joined with suitable internucleoside groups. The term “monomer” refers to any chemical group that can be incorporated within a polynucleotide chain, including natural nucleotides and non-nucleotides capable of being linked through internucleoside linkages. Nucleotide monomers typically include a nucleobase or simply a “base.” Polynucleotides of the invention include those having modified bases such as are disclosed, for example, in U.S. Pat. No. 6,001,651. The term “polynucleotide” encompasses heteropolymers containing nonnucleotide monomers or monomers having modified bases. In the polynucleotide deoxyribopolynucleotide, the internucleoside groups joining the nucleotides are phosphodiester groups. Other suitable joining groups include, but are not limited to, phosphorothioates, methylphosphonates, phosphorodithioates, phosphoroamidates, carbamates, amides, and sulfones. Polynucleotides of the invention can contain two or more different types of internucleoside groups.

An “oligonucleotide” or “oligomer” is a polynucleotide with a length of less than about 100 nucleotides.

An “m5CG sequence” is a dinucleotide sequence with a 5′ 5-methylcytidine residue and a 3′ guanosine residue. “m5CpG” denotes a dinucleotide having an internucleoside phosphate linkage.

An “m5CN1G sequence” and an “m5CN2G sequence” both refer to a trinucleotide sequence with a 5′ 5-methylcytidine residue linked to a nucleoside, which is linked to a 3′ guanosine. In “5CpN1pG,” both internucleoside linkages are phosphate linkages. N1 can be any nucleoside; whereas, N2 is complementary to N1. N3 can be any nucleotide. N4 can be any nucleotide.

An “unmethylated CG sequence” or “unmethylated CNG” contains cytidine in place of 5-methylcytidine.

Polynucleotides are said to be “complementary” if they are capable of hybridizing with one another sufficiently well and with sufficient specificity, to give the desired effect. In the context of the invention, “hybridization” refers to hydrogen bonding, which may be Watson-Crick, Hoogsteen or reversed Hoogsteen hydrogen bonding, between complementary nucleotide bases. For example, adenine and thymine are complementary bases that pair through the formation of hydrogen bonds. Thus, as used herein, the term “complementary,” refers to the capacity for precise pairing between two nucleotides. If a nucleotide at a certain position of an oligonucleotide is capable of hydrogen bonding with a nucleotide at the same position of another polynucleotide, then the oligonucleotide and the polynucleotide are considered to be complementary to one another at that position. The oligonucleotides and target nucleotide sequences of the invention are complementary to each other when a sufficient number of corresponding positions in each molecule are occupied by nucleotides that hydrogen bond with each other. Thus, “complementary” is used herein to indicate a sufficient degree of precise pairing such that stable and specific binding occurs between the oligonucleotide and molecule having a complementary nucleotide sequence. It is understood in the art that the sequence of a first oligonucleotide need not be 100% complementary to that of another to hybridize.

An oligonucleotide is said to be “specific for” a target nucleotide sequence when a) the oligonucleotide binds to the target nucleotide sequence with sufficient affinity to form a complex with the target nucleotide, and b) there is a sufficient degree of complementarity to avoid significant non-specific binding of the oligonucleotide to a non-target sequence under conditions in which specific binding is desired. In the case of in vivo assays or therapeutic applications, oligonucleotides of the invention are selected to minimize non-specific binding under physiological conditions. In the case of in vitro assays, oligonucleotides are selected to minimize non-specific binding under the assay conditions.

DNA binding and methylator segments of an oligonucleotide are to be “operably linked” if the methylator segment is able to induce methylation at a target nucleotide sequence complementary to the DNA binding element. Operably linked elements can be directly adjacent to one another or can be separated by one or more monomers or other elements.

The phrase “methylation at a target nucleotide sequence” refers to methylation of one or more nucleotides within or in the vicinity of the target nucleotide sequence. Where the target nucleotide sequence is present in a double-stranded polynucleotide, one or more nucleotides on either strand (or both strands) of the polynucleotide can become methylated. Methylation can be monitored by any convenient method for determining the degree of methylation at a target nucleotide sequence (see, for example, Vu, T. H. et al. (2000) Genomics 64:132-143, describing bisulfite genomic sequencing, and Hu, J. F. et al. (1998) Molecular Endocrinology 12:220-232, describing Southern blotting). Gene methylation also can be monitored indirectly by assaying expression of a target gene as illustrated below in Examples 1, 2, 4 and 5. Generally, methylation occurs within about 5 kilobases (kb) of the target nucleotide sequence. In alternative embodiments, methylation occurs within about 2 kb, about 1 kb, or about 500 basepairs (bp) of the target nucleotide sequence.

As used herein, the term “gene” refers to all nucleotide sequences associated with a gene, including coding sequences; non-coding sequences such as 5′ and 3′ untranslated regions and introns, as well as any other sequences containing elements that regulate transcription of the gene, such as promoter regions. The “template” strand of a gene is used to transcribe RNA in a reverse (non-sense) direction. The sense strand of DNA is complementary to the template strand. The target DNA sequence can be on either strand of DNA.

The phrase “gene regulatory region” refers to regions including nucleotide sequences containing elements that regulate transcription of a gene, including but not limited to promoters, enhancers, splicing sites, 5′-regulatory or 3′-regulatory regions, suppressors, and silencers.

As used herein, the terms “disease” and “disorder” refer to any condition of an organism that impairs normal physiological functioning.

As used herein, “a disease gene” is any gene whose expression or overexpression correlates with a disease or disorder.

The term “cancer” refers to any disease characterized by uncontrolled cell growth.

The term “normal organism” is used herein to refer to an organism that has not been subjected to the targeted methylation of the invention.

Introducing an oligonucleotide into a cell “in vivo” refers to introducing the oligonucleotide into a cell when it is part of a multicellular organism. As used in this context, the term “ex vivo” refers to introducing the oligonucleotide into a cell when it is not part of a multicellular organism. The term “ex vivo” encompasses introducing an oligonucleotide into a cell, e.g., for research applications, as well as introducing an oligonucleotide into a cell that is then delivered to an organism, e.g., for therapeutic applications.

Oligonucleotides may also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-me pyrimidines, particularly 5-methylcytosine (also referred to as 5-methyl-2′deoxycytosine and often referred to in the art as 5mC), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N⁶ (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine, may be included. m5C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278) and are presently preferred base substitutions.

In one embodiment, the invention provides an oligonucleotide capable of inducing methylation at a target nucleotide sequence. The polynucleotide includes an oligonucleotide methylator segment operably linked to a DNA binding segment.

An oligonucleotide “methylator segment” is a double-stranded oligonucleotide that enhances the efficiency of methylation of a DNA sequence in the vicinity of the methylator segment. A methylator segment according to the invention is an oligonucleotide duplex having a first strand and a second strand complementary to the first strand. The first strand is part of the DNA binding segment that is complementary to the target sequence or its complementary sequence. In one embodiment, the methylator segment has at least one m5CG sequence on the second strand paired with an unmethylated CG sequence on the first strand, thus forming a hemi-methylated duplex. Two strands may be linked by a short loop composed of one or more oligonucleotides. In another embodiment, the methylator segment has at least one 5′-m5CN1G-3′ sequence on the first strand paired with an unmethylated 5′-CN2G-3′ sequence, wherein N1 is any nucleotide, and N2 is a nucleotide that pairs with N1. In preferred embodiments, the internucleoside linkages in the methylator elements of the invention are phosphate-based, for example, phosphodiester or phosphorothioate groups. Thus, preferably, the m5CG sequence is an m5CpG sequence, the CG sequence is a CpG sequence, the m5CN1G sequence is an m5CpN1pG sequence, and the m5CN2G sequence is an m5CpN2pG sequence.

In one embodiment, the first strand includes a 5′-Gm5CG-3′ sequence, and the second strand includes a corresponding unmethylated 5′-CGC-3′ sequence, thus forming a duplex having a methylated site in the first strand. Alternatively, the first strand can include an unmethylated 5′-GCG-3′ sequence, with the second strand including a methylated 5′-m5CGC-3′ sequence, thus forming a similar hemi-methylated structure but with the methylated 5′-m5CGC-3′ in the second strand.

The first strand and the second strand of the methylator element can form a double helical structure through hydrogen bonding, which, in preferred embodiments, is Watson-Crick hydrogen bonding. The stability of the double helical structure is related to the length of the first and second strands as well as the presence or absence of a linkage between the strands. The relative stability of a duplex can be assessed by determining the duplex melting temperature. Also, it is possible to predict duplex stability using algorithms such as those of Owczarzy, R. et al. (Biopolymers 1997; 44(3):217-39).

If desired, the first and second strands of the methylator segment can be linked covalently to further stabilize the methylator segment. The 5′ end of the first strand can be linked to the 3′ end of the second strand or vice versa. In addition to end-to-end linkage, the two strands can be attached via a linkage internal to either or both of the strands. In an example of end-to-end linkage, the first and second strands can be linked by one or more nucleotides. Such nucleotide(s) can form a loop connecting the first and second strands, so that the methylator segment forms a so-called hairpin structure.

Parameters for the design of hairpin structures are well known and include the length and sequence of the loop. For example, Vallone et al. (Vallone, P. M. et al. (1999) Biopolymers 50:425-42) teach the optimization of tetraloop sequences. The influence of loops on the stability of DNA duplexes has been studied extensively (see, e.g., Senior, M. M. et al. (1988) Proc. Natl. Acad. Sci. USA 85(17):6242-6; Xodo, L. E. (1988) Polynucleotides Res. 16(9):3671-91), and thus those skilled in the art can readily design hairpin structures suitable for use in the methylator segments of the invention.

In one embodiment, the linker joining the first and second strands of the methylator segment is a single nucleotide, for example, a single thymine. A preferred methylator segment of this type has the sequence of 5′-Gm5CG-T-CGC-(N)n-3′ (SEQ ID NO: cc) with the linker thymine shown in bold).

In addition to the methylator sequence given above, other useful structures include but are not limited to Tm5CG-Y-CGA (SEQ ID NO: ), Cm5CG-Y-CGG (SEQ ID NO: ), and Am5CG-Y-CGT (SEQ ID NO: ), where Y is a suitable linker. A general form of the methylator sequence is (G/C/T/A)m5CG-Y-CG(C/G/A/T) (SEQ ID NO: ) or N1m5CG-Y-CGN2 (SEQ ID NO: ), where N1 is any base and N2 is complementary to N1.

Non-nucleotide linkers also are useful for covalently linking the first and second strands of the methylator segment. The linker can be, for example, an aliphatic linker, joined to one end of the first strand and an end (usually the closest end) of the other strand. Suitable linkers of this type include, for example, those disclosed by Pils, W. and Micura, R. (2000) Nucleic Acids Res. 28:1859-1863 and by Durand, M. et al. (1990) Nucleic Acids Res. 18(21):6353-9. Polyethylene and polyethylene glycol linkages can also be employed, as can rigid linkers suitable for hairpin formation, including, for example, stilbene linkers (Nelson, J. S. et al. (1996) Biochemistry 35:5339-44).

As noted above, linkers can connect the first and second strands of the methylator segment at interior positions such that a linker extends from a base or internucleoside unit on one strand to a base or internucleoside unit on the opposing strand. Alkyl linkers, such as those described by Gao et al. ((1995) Polynucleotides Res. 23:285-92), for example, can be employed in the methylator segment to join phosphorothioate internucleoside groups on each strand.

The first and second strands of a methylator segment also can be linked multiple times to form a macrocylic structure. Cyclization of oligonucleotides is known to one of skill in the art (see, for example, Kool, E. T. (1996) Annu. Rev. Biophys. Biomol. Struct. 25:1-28) and can readily be employed in the methylator elements of the invention.

DNA Binding Segment

The second strand of the methylator segment is operably linked to a single-stranded oligonucleotide DNA binding segment that is complementary to a target nucleotide sequence in a polynucleotide region to be methylated. The DNA binding segment can be complementary either to the template or the complementary strand of the target DNA sequence. The DNA binding segment of the invention includes at least one m5CG sequence or at least one m5CN3G sequence, wherein N3 can be any nucleotide. In a preferred embodiment, a DNA binding segment has multiple m5C nucleotides. In general, any C residue in the DNA binding segment can be substituted with an m5C residue. In preferred embodiments, the internucleoside groups are phosphate-based, including, for example, phosphodiester or phosphorothioate groups.

A DNA binding segment has any length that allows specific hybridization to the target nucleotide sequence, preferably from about 8 to about 50 nucleotides, more preferably from about 12 to about 30, and most preferably from about 16 to about 20 nucleotides in length. The length of the DNA binding segment is chosen such that the DNA binding segment can stably and specifically recognize the target nucleotide sequence. A DNA binding segment usually, but not necessarily, has at least about 16 nucleotides in order to recognize a specific site within a genome. However, because of repetition of DNA sequences, it may be preferable to choose a somewhat longer sequence. Also, because the length of the sequence also affects the stability of the complex formed with the target nucleotide sequence, longer sequences are generally preferred. To reach a maximal silencing effect, the target sequence is usually chosen from a region that contains multiple CpG dinucleotides (also called a CpG island) in a promoter, enhancer, exon, intron, splicing site, silencer, repressor, and 5′-regulatory or 3′-regulatory region.

The DNA binding segment actually overlaps with the methylator segment, the first strand of the methylator segment is also capable of hybridizing with the target sequence. The DNA binding and methylator segments can be linked end-to-end or via a linkage internal to one or both elements. In end-to-end linkage, the 5′ end of the DNA binding segment can be linked to the 3′ end of the methylator segment or vice versa. In one embodiment, the DNA binding and the methylator segments are directly linked by an internucleoside group, such as those described above. In another embodiment, the DNA binding element and the methylator segments are operably linked with one or more monomers, preferably one or more nucleotide monomers, such as thymidine. In addition, any of the alternative linkages described above for linking the first and second strands of the methylator segment can be employed to link the DNA binding segment to the methylator segment.

In an exemplary embodiment, the silencing compound is a 21 nucleotide oligomer having the sequence 5′-Gm5CGTCGCAGm5CGCTGAGTm5CGGT-3′ (SEQ ID NO: ), which targets human tumor necrosis factor alpha (TNFα). In this silencing compound, the methylator segment (5′-Gm5CG-3′) is linked to 5′-CGC-3′ in the DNA binding segment to form a semi-methylated hairpin via a single T nucleotide. This silencing compound directs methylation at a target nucleotide sequence in human TNFα and silences the gene when introduced in the cell.

A DNA binding segment can also be directed at other genes, including pathogenic genes. For a given gene target, several DNA binding segments can be synthesized with an appropriate methylator segment. The resulting drug candidates can then be easily tested for inactivation of the gene target using the screening assays described herein or screening assays known to those of skill in the art for the expression of the target gene. Exemplary DNA binding segments for several exemplary genes include those described in the following tables.

TABLE 1

Silencing compounds for the human interleukin-6
(IL-6) gene

| | | |
|---|---|---|
| No: | Silencing compounds | SEQ ID NO |

| | | |
|---|---|---|
| IL6-01: | Gm5CGACGCAACTGGACm5CGAAG | aa |
| IL6-02: | Tm5CGTCGAGGATGTACm5CGAAT | aa |
| IL6-03: | Tm5CGCCGAGATGC5mCGTm5CGAGGAT | aa |
| IL6-04: | CGCTGm5CGCAGAATGAGATG | aa |
| IL6-05: | TCGCm5CGAAGAGCCCTCAGGCT | aa |
| IL6-06: | Am5CGTCGTGTCCTAAm5CGCTCAT | aa |
| IL6-07: | Cm5CGTCCGAGGTGCCCATGCTA | aa |
| IL6-08: | TCGGCTm5CGAGGGCAGAATGAGC | aa |
| IL6-09: | CGCAGm5CGCTCGACGCm5CGCTGGCA | aa |
| IL6-10: | CGGACm5CGAAGGCGCCTGTGCm5CGGA | aa |
| IL6-11: | ACGTm5C; GTCGAGGATGTACm5CGA | aa |
| IL6-12: | ACGTTm5CGTCAATTm5CGTTCTGA | aa |

TABLE 2

Silencing compounds for the human CDC25A gene

| | | |
|---|---|---|
| No: | Silencing compounds | SEQ ID NO |

| | | |
|---|---|---|
| CDC25-1: | TGm5CGGACCCTCCAGGCGCTGm5CG | bb |
| CDC25-2: | Tm5CGACGACTCCGm5CGGTTCAG | |
| CDC25-2B: | Tm5CGACGACTCCGm5CGGTTCAGG | |
| CDC25-3: | Gm5CGTCGCAGAGCTCCm5CGCT | |
| CDC25-3B: | TGm5CGGACCCTCCAGGCGCTGm5CG | |
| CDC25-4: | Cm5CGTCGGGCCCAGTTCCATG | |
| CDC25-5: | Gm5CGTCGCCTTCACGAm5CGGGCT | |
| CDC25-6: | Gm5CGTCGCCAAATAGm5CGCCTTC | |
| CDC25-7: | Am5CGTCGTCCATAGTGAm5CGGTC | |
| CDC25-8: | Cm5CGTCGGCAACCAAGCTGTA | |
| CDC25-8B: | Cm5CGTCGGCAACCAGCTGTAAG | |

TABLE 3

Silencing compounds for the human MKP-1 gene

| | | |
|---|---|---|
| No: | Silencing sequences | SEQ ID NO |

| | | |
|---|---|---|
| MKP-01: | Gm5CGTCGCAGGCCTCCAGm5CGTC | cc |
| MKP-02: | Gm5CGGCAGTCCAGCCGCAGm5CG | |
| MKP-02B: GAGm5CGGCAGTCCAGCCGCAGm5CG | | |
| MKP-03: | Gm5CGTCGCACGTTGACAGAGCm5CG | |
| MKP-04: | Gm5CGTCGCACGATGGTGCTGA | |
| MKP-05: | Tm5CGACGATGTGCTCCAGGC | |
| MKP-06: | Gm5CGTCGCGGAGCTCGGm5CGTTG | |
| MKP-07: | m5CGCTGCGCTm5CGTCCAGCA | |
| MKP-08: | Gm5CGTCGCTGTCAGGGAm5CGCT | |
| MKP-09: | Gm5CGACGCACTGCCCAGGTAC | |
| MKP-09B: | Gm5CGACGCACTGCCCAGGTACAG | |
| MKP-10: | Am5CGTCGTCCAGCTTGACTm5CG | |
| MKP-11: | Tm5CGTCGAGCACAGCCATGGC | |
| MKP-11B: | Tm5CGTCGAGCACAGCCATGGm5CGG | |

TABLE 4

Silencing compounds for the human B-cell lymphoma-
2 (Bcl-2) gene

| | | |
|---|---|---|
| No: | Silencing sequences | SEQ ID NO |

| | | |
|---|---|---|
| Bc12-01 | Tm5CGACGACCGTGGCAAAGm5CGT | dd |
| Bc12-01B | Tm5CGCGACCGTGGCAAAGm5CGTC | |
| Bc12-02 | Cm5CGACGGGm5CGTCAGGTGCAGC | |
| Bc12-02B | Cm5CGACGGGm5CGTCAGGTGCAGCT | |
| Bc12-03 | Gm5CGTCGCGGCGGTAGCGGm5CG | |
| Bc12-04 | Gm5CGACGCm5CGTCCCTGAAGAGCT | |
| Bc12-05 | Am5CGTCGTACAGTTCCACAAAG | |
| Bc12-06 | Gm5CGTCGCATCCCACTm5CGTAG | |
| Bc12-07 | Tm5CGACGAAGGCCACAATCCT | |
| Bc12-08 | Tm5CGACGACATCTCCm5CGGTTGA | |
| Bc12-09 | CGCAGm5CGTGm5CGCCATCCTTC | |
| Bc12-10 | CACAATCm5CGCCCCCCAGTm5CTG | |
| Bc12-11 | Gm5CGACGCTCTCCAm5CGCACAT | |
| Bc12-12 | Am5CGTCGTTATCm5CTGGATCCA | |
| Bc12-13 | GCATCCCAGCCTCCGTTAm5CG | |

TABLE 5

Silencing compounds for the human TNFαgene

| | | |
|---|---|---|
| No: | Silencing sequences | SEQ ID NO |

| | | |
|---|---|---|
| TNFkex-01 | GCACm5CGCCTGGAGCCGTTAm5CG | ee |
| TNFkex-02 | AGTm5CGAGATAGTCGGGCm5CGA | |
| TNFkex-03 | AGTm5CGAGATAGTCGGGCm5CG | |
| TNFkex-04 | Gm5CGTCGCCTGCCAm5CGATCAG | |
| TNFkex-05 | Gm5CGTCGCAGm5CGCTGAGTm5CGGT | |
| TNFkex-06 | Tm5CGTCGATTGATCTCAGm5CGCT | |
| TNFkex-07 | Cm5CGTCGGTTCAGCCAm5CTGGAG | |
| TNFkex-08 | ACGGGm5CGATGTGGm5CGTCTGAG | |
| TNFkex-09 | Am5CGACGTCCm5CGGATCATGCTTTC | |
| TNFkex-10 | Cm5CGTCGGTCAGTATGTGAGA | |
| TNFkex-11 | GGCTGCm5CGATCACTCCAAAGTGC | |
| | TGm5CGGACCCTCCAGGCGCTGm5CG, | |
| | Tm5CGACGACTCCGm5CGGTTCAGG, Cm5CGTCGGCAACCAGCTGTAAG, | |

The target nucleotide sequence can be in a gene, the expression of which is to be down regulated, and is preferably in a regulatory region of the gene. Any gene in any organism wherein methylation could regulate gene expression can be targeted. Thus, the target nucleotide sequence can be one present in a eukaryotic, prokaryotic (e.g., E. coli), or plant gene. In preferred embodiments the target nucleotide sequence is a mammalian gene sequence, more preferably, a target sequence present in a mammal having research or commercial value, such as for example, a mouse, rat, cat, dog, or monkey or a chicken, pig, sheep, goat, cow, or horse. In a particularly preferred embodiment, the target nucleotide sequence is a human gene sequence.

Exemplary regulatory regions that can be targeted include regions, including, but not limited to, promoter, enhancer, intron, exon, 5′-end and 3′-end, splicing site, silencer, suppressor, and imprinting center. Target nucleotide sequences can be located, for example, near a TATA box, CpG-rich regions, or the binding site of a transcription factor, such as Sp1, and the like. Target nucleotide sequences useful in the invention have at least one CpG or CpN1pG sequence, wherein N1 can be any nucleotide. Preferred target nucleotide sequences are in CpG rich regions, such as CpG islands, or CpN1pG rich regions. Generally, several nucleotide targets can be tested as described in Example 1 and 2 to identify a target nucleotide sequence that gives the desired results for a particular application.

In preferred embodiments, the gene targeted is a disease gene, such as a gene associated with cancer or another abnormal cellular function. Numerous genes have been shown to play a role in the etiology of various cancers. “Oncogenes” is the term given to those genes whose overexpression or inappropriate expression plays a role in the initiation or progression of cancer. Any oncogene or proto-oncogene can be targeted for methylation according to the present invention. Exemplary oncogenes include those encoding growth factors or growth factor receptors. In one embodiment, the target nucleotide sequence is in a gene that is normally imprinted (hypermethylated) but becomes hypomethylated in cancer cells. An embodiment is illustrated herein using the Igf2 gene, which is hypomethylated in liver cancers. An oligonucleotide of the invention designed to target the Igf2 gene promoter was shown to inhibit expression of the Igf2 gene (see Examples 1, 2 and 5). Example 6 shows that in a mouse model of liver cancer, a composition including this oligonucleotide reduces tumor growth in vivo.

Compositions

The invention also provides compositions including the oligonucleotides of the invention and at least one other component, such as a storage solution (e.g., a suitable buffer), a component that facilitates entry of the oligonucleotide into a cell, and/or a physiologically acceptable carrier.

Components that facilitate intracellular delivery of oligonucleotides are well known and include, but are not limited to, lipids, liposomes, water-oil emulsions, polyethylene imines and dendrimers, any of which can be used in compositions according to the invention.

Liposomes fall into two broad classes. Cationic liposomes are positively charged liposomes which interact with negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

Liposomes which are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g. as a solution or as an emulsion) were ineffective (Weiner et al., Journal of Drug Targeting, 1992, 2, 405-410). Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18, 259-265).

Non-ionic liposomal systems have also been examined to determine their utility in the delivery of drugs to the skin, in particular systems comprising non-ionic surfactant and cholesterol. Non-ionic liposomal formulations comprising Novasome™ I (glyceryl dilaurate/cholesterol/polyoxyethylene-10-stearylether) and Novasome™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporin-A into different layers of the skin (Hu et al. S.T.P.Pharma. Sci., 1994, 4, 6, 466).

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside Gm1, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765). Various liposomes comprising one or more glycolipids are known in the art. Papahadjopoulos et al. (Ann. NY Acad. Sci., 1987, 507, 64) reported the ability of monosialoganglioside Gm1, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both to Allen et al., disclose liposomes comprising (1) sphingomyelin and (2) the ganglioside Gm1 or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al.).

Many liposomes comprising lipids derivatized with one or more hydrophilic polymers, and methods of preparation thereof, are known in the art.

Liposomes containing oligonucleotides are prepared by known methods, such as, for example, those described in Epstein, et al. (1985) PNAS USA 82:3688-3692, and Hwang, et al. (1980) PNAS USA, 77:4030-4034. Ordinarily the liposomes in such preparations are of the small (about 200-800 Angstroms) unilamellar type in which the lipid content is greater than about 30 mol. percent cholesterol, the specific percentage being adjusted to provide the optimal therapy. Useful liposomes can be generated by the reverse-phase evaporation method, using a lipid composition including, for example, phosphatidylcholine, cholesterol, and PEG-derivatized phosphatidylethanolamine (PEG-PE). If desired, liposomes are extruded through filters of defined pore size to yield liposomes of a particular diameter.

In another embodiment, compositions of the invention include dendrimers complexed to oligonucleotides which can be used to transfect cells. Suitable dendrimers include, but are not limited to, “starburst” dendrimers and various dendrimer polycations. Dendrimer polycations are three dimensional, highly ordered oligomeric and/or polymeric compounds typically formed on a core molecule or designated initiator by reiterative reaction sequences adding the oligomers and/or polymers and providing an outer surface that is positively charged. Such dendrimers may be prepared as disclosed in PCT/US83/02052, and U.S. Pat. Nos. 4,507,466; 4,558,120, 4,568,737; 4,587,329; 4,631,337; 4,694,064; 4,713,975; 4,737,550; 4,871,779; and 4,857,599.

Dendrimer polycations are preferably non-covalently associated with the oligonucleotides of the invention. This permits an easy disassociation or disassembling of the composition once it is delivered into the cell. Typical dendrimer polycations suitable for use herein have a molecular weight ranging from about 2,000 to 1,000,000 daltons (Da), and more preferably about 5,000 to 500,000 Da. However, other molecular weights can also be employed. Preferred dendrimer polycations have a hydrodynamic radius of about 11 to 60 angstroms (A.), and more preferably about 15 to 55 A. Other sizes, however, are also suitable for use in the invention. Methods for the preparation and use of dendrimers to introduce oligonucleotides into cells in vivo are well known to those of skill in the art and described in detail, for example, in U.S. Pat. No. 5,661,025.

Compositions of the invention can be tested for their ability to deliver oligonucleotides into cultured cells by any convenient assay, such as the fluorescent assay described below in Example 3.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. Pharma. Sci., 1977, 66, 1-19). The base addition salts of the acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention.

As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 α-amino acids involved in the synthesis of proteins in nature, for example, glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation.

Suitable pharmaceutically acceptable cations are well known to those skilled in the art and include alkaline, alkaline earth, ammonium and quaternary ammonium cations. Carbonates or hydrogen carbonates are also possible.

For oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example, hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The present invention also includes pharmaceutical compositions and formulations which include the antisense compounds of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions of the invention can include a physiologically acceptable carrier, excipient, or stabilizer, such as those described in Remington: the Science and Practice of Pharmacy (2000) 20th edition, Oslo, ed. by A. R. Gennaro. A physiologically acceptable carrier, excipient, or stabilizer suitable for use in the invention is non-toxic to cells or recipients at the dosages employed, and can include a buffer (such as a phosphate buffer, citrate buffer, and buffers made from other organic acids), an antioxidant (e.g., ascorbic acid), low-molecular weight (less than about 10 residues) polypeptide, a protein (such as serum albumin, gelatin, and an immunoglobulin), a hydrophilic polymer (such as polyvinylpyrrolidone), an amino acid (such as glycine, glutamine, asparagine, arginine, and lysine), a monosaccharide, a disaccharide, and other carbohydrates (including glucose, mannose, and dextrins), a chelating agent (e.g., ethylenediaminetetracetic acid [EDTA]), a sugar alcohol (such as mannitol and sorbitol), a salt-forming counter ion (e.g., sodium), and/or an anionic surfactant (such as Tween® and Pluronic® nonionic surfactants and PEG). In one embodiment, the physiologically acceptable carrier is an aqueous pH-buffered solution.

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate oligonucleotide in hepatic tissue can be reduced when it is coadministered with polyinosinic acid, dextran sulfate, polycytidic acid or 4-acetamido-4′isothiocyano-stilbene-2,2′-disulfonic acid (Miyao et al., Antisense Res. Dev., 1995, 5, 115-121; Takakura et al., Antisense & Nucl. Acid Drug Dev., 1996, 6, 177-183).

In contrast to a carrier compound, a “pharmaceuticalcarrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition.

Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipient suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

The compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances which increase the viscosity of the suspension including, for example, sodium carboxymethylcellulose, sorbitol and/or dextran. The suspension may also contain stabilizers. Certain embodiments of the invention provide pharmaceutical compositions containing (a) a methylating compound and (b) one or more other chemotherapeutic agents which function by a methylating mechanism. Examples of such chemotherapeutic agents include, but are not limited to, anticancer drugs such as daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin and diethylstilbestrol (DES). See, generally, THE MERCK MANUAL OF DIAGNOSIS AND THERAPY, 17th Ed., Beers et al., eds., 1999, Rahway, N.J., pages 973-1000). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. See, generally, THE MERCK MANUAL OF DIAGNOSIS AND THERAPY, 17th Ed., Beers et al., eds., 1999, Rahway, N.J., pages 1001-1322 and 46-49, respectively). Other chemotherapeutic agents are also within the scope of this invention. Two or more combined compounds may be used together or sequentially.

For prophylactic or therapeutic use, oligonucleotides of the invention are formulated in a manner appropriate for the particular indication. U.S. Pat. No. 6,001,651 to Bennett et al. describes a number of pharmaceutical compositions and formulations suitable for use with an oligonucleotide therapeutic as well as methods of administering such oligonucleotides. In a preferred embodiment, prophylactic or therapeutic compositions of the invention include oligonucleotides combined with lipids, as described above.

Compositions of the invention can be stored in any standard form, including, e.g., an aqueous solution or a lyophilized cake. Such compositions are typically sterile when administered to cells or recipients. Sterilization of an aqueous solution is readily accomplished by filtration through a sterile filtration membrane. If the composition is stored in lyophilized form, the composition can be filtered before or after lyophilization and reconstitution.

The compositions of the present invention may be prepared and formulated as emulsions. Emulsions are typically heterogenous systems of one liquid dispersed in another in the form of droplets usually exceeding 0.1 μm in diameter. (Idson, in PHARMACEUTICAL DOSAGE FORMS, Lieberman, Rieger and Banker (Eds.), 1988, Marcel Dekker, Inc., New York, N.Y., volume 1, p. 199; Rosoff, in PHARMACEUTICAL DOSAGE FORMS, Lieberman, Rieger and Banker (Eds.), 1988, Marcel Dekker, Inc., New York, N.Y., Volume 1, p. 245; Block, in PHARMACEUTICAL DOSAGE FORMS, Lieberman, Rieger and Banker (Eds.), 1988, Marcel Dekker, Inc., New York, N.Y., volume 2, p. 335; Higuchi et al., in REMINGTON'S PHARMACEUTICAL SCIENCES, Mack Publishing Co., Easton, Pa, 1985, p. 301). Emulsions are often biphasic systems comprising of two immiscible liquid phases intimately mixed and dispersed with each other. In general, emulsions may be either water-in-oil (w/o) or of the oil-in-water (o/w) variety. When an aqueous phase is finely divided into and dispersed as minute droplets into a bulk oily phase the resulting composition is called a water-in-oil (w/o) emulsion. Alternatively, when an oily phase is finely divided into and dispersed as minute droplets into a bulk aqueous phase the resulting composition is called an oil-in-water (o/w) emulsion. Emulsions may contain additional components in addition to the dispersed phases and the active drug that may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulationsoften provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous provides an O/W/O emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in PHARMACEUTICAL DOSAGE FORMS, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (Rieger, in PHARMACEUTICAL DOSAGE FORMS, Lieberman, Rieger and Banker (Eds.), 1988, Marcel Dekker, Inc., New York, N.Y., volume 1, p. 285; Idson, in PHARMACEUTICAL DOSAGE FORMS, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric (Rieger, in PHARMACEUTICAL DOSAGE FORMS, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in PHARMACEUTICAL DOSAGE FORMS, Lieberman, Rieger and Banker (Eds.), 1988, Marcel Dekker, Inc., New York, N.Y., volume 1, p. 335; Idson, in PHARMACEUTICAL DOSAGE FORMS, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (Idson, in PHARMACEUTICAL DOSAGE FORMS, Lieberman, Rieger and Banker (Eds.), 1988, Marcel Dekker, Inc., New York, N.Y., volume 1, p. 199). Emulsion formulations for oral delivery have been very widely used because of reasons of ease of formulation, efficacy from an absorption and bioavailability standpoint. (Rosoff, in PHARMACEUTICAL DOSAGE FORMS, Lieberman, Rieger and Banker (Eds.), 1988, Marcel Dekker, Inc., New York, N.Y., volume 1, p. 245; Idson, in PHARMACEUTICAL DOSAGE FORMS, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

In one embodiment of the present invention, the compositions of oligonucleotides are formulated as microemulsions. A microemulsion may be defined as a system of water, oil and amphiphile which is a single optically isotropic and thermodynamically stable liquid solution (Rosoff, in PHARMACEUTICAL DOSAGE FORMS, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: CONTROLLED RELEASE OF DRUGS: POLYMERS AND AGGREGATE SYSTEMS, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in REMINGTON'S PHARMACEUTICAL SCIENCES, Mack Publishing Co., Easton, Pa., 1985, p. 271).

Methods

The invention includes a method of using the silencing compounds of the invention for inducing methylation at a target nucleotide sequence in a cell. Because the oligonucleotides of the invention can be directed against any target nucleotide sequence, this method has wide application in research, diagnostics, prophylaxis, and therapeutics.

In one embodiment, the method of the invention includes introducing an oligonucleotide of the invention into a cell, thereby methylating a target nucleotide sequence, and then determining a phenotypic change associated with the methylation at the target nucleotide sequence. In research applications the target nucleotide sequence can be in a gene (preferably in its regulatory region) encoding a protein of unknown function. Protein function can be studied in cultured cells or in “knock-out” organisms by methylating the DNA sequence of interest and identifying an associated change in phenotype.

Examples 1 and 2 illustrate how an oligonucleotide of the invention can be used to methylate the Igf2 gene in cultured cells. Conventional knock-out organisms, typically knock-out mice, are well known and are typically produced by substituting a defective gene for the native gene, e.g., by homologous recombination. Knock-out organisms of the present invention, by contrast, retain the native gene, which is either not expressed or expressed at a reduced level due to methylation at a target sequence within the gene, preferably within the gene regulatory region. Thus, references herein to “inactivation of a gene” refer to the inhibition of expression of a gene, as opposed to its physical disruption. The production of knock-out organisms is described below with respect to producing a knock-out animal. However, one skilled in the art can readily apply these teachings to other organisms.

The present invention encompasses knock-out animals that have a particular gene inactivated in one tissue, a plurality of tissues, or all tissues. To produce a knock-out animal having a gene inactivated in one tissue, the silencing compound is preferably introduced into a progenitor cell for that tissue. To produce a knock-out animal having a gene inactivated in a plurality of tissues, it is preferable to introduce the silencing compound into a pluripotent cell that gives rise to the tissues for which gene inactivation is desired. Generally, it is preferable to produce a knock-out animal having a gene inactivated in all cells, which is conveniently accomplished by introducing the silencing compound into a totipotent cell. Totipotent cells are capable of giving rise to all cell types of an embryo, including germ cells. Depending on when gene inactivation occurs, chimeric animals (i.e., those wherein the gene is active in some cells and not in others) can also be produced from totipotent cells.

Totipotent embryonic stem cell lines (“ES” cells) have been isolated by culturing cells derived from very young embryos (blastocysts) (Evans, et al. (1981) Nature, 292:154-156; Bradley, et al. (1984) Nature, 309:255-256; Gossler, et al. (1986) Proc. Natl. Acad Sci USA 83:9065-9069; and Robertson, et al. (1986) Nature, 322:445-448). Such cells are capable, upon incorporation into an embryo, of differentiating into all cell types, including germ cells, and can be employed to generate animals in which the expression of a particular gene is suppressed. Alternatively, a silencing compound can be introduced into the nucleus of one cell which can then be transferred to a fertilized egg using the conventional nuclear transfer techniques that have been employed to clone animals.

Any ES cell may be used in accordance with the present invention. It is, however, preferred to use primary isolates of ES cells. Such isolates may be obtained directly from embryos such as the CCE cell line disclosed by Robertson, E. J.: CURRENT COMMUNICATIONS IN MOLECULAR BIOLOGY, Capecchi, M. R. (ed.), Cold Spring Harbor Press, Cold Spring Harbor, N.Y. (1989), pp. 39-44, or from the clonal isolation of ES cells from the CCE cell line (Schwartzberg et al. (1989) Science 212: 799-803). Such clonal isolation can be accomplished according to the method of Robertson (1987) In: TERATOCARCINOMAS AND EMBRYONIC STEM CELLS: A PRACTICAL APPROACH, E. J. Robertson, Ed., IRL Press, Oxford. The purpose of such clonal propagation is to obtain ES cells that differentiate into an animal with great efficiency. Example of ES cell lines which have been clonally derived from embryos are the ES cell lines known as AB I (hprt⁺) or AB2.1 (hprt⁻).

The ES cells are preferably cultured on stromal cells (such as STO cells (especially SNL76/7 STO cells) and/or primary embryonic G418 R fibroblast cells) as described by Robertson, supra. The stromal (and/or fibroblast) cells serve to eliminate the clonal overgrowth of abnormal ES cells. Most preferably, the cells are cultured in the presence of leukocyte inhibitory factor (“lif”) (Gough et al. (1989) Reprod. Fertil. Dev. 1:281-288; Yamamori et al. (1989) Science, 246:1412-1416). Since the gene encoding lif has been cloned (Gough, et al. supra.), it is especially preferred to transform stromal cells with this gene by means known in the art, and then to culture the ES cells on transformed stromal cells that secrete lif into the culture medium.

ES cell lines useful in the invention can be derived from any species (for example, chicken, etc.), although cells derived or isolated from mammals such as rodents, rabbits, sheep, goats, pigs, cattle, primates and humans are preferred. Cells derived from rodents (i.e. mouse, rat, hamster, etc.) are particularly preferred.

Once a silencing compound has been introduced into a totipotent cell, the cell is implanted into the uterus of a recipient female and allowed to develop. If instead the oligonucleotide is introduced into a progenitor cell or a pluripotent cell, the cell is introduced into an embryo of the appropriate stage, which is then implanted.

Silencing compounds can also be used for the study of gene methylation and demethylation processes within a cell. For example, an oligonucleotide of the invention can be used to methylate a target nucleotide sequence, which can then serve as a substrate in the study of demethylation processes and/or be used for the study of methylation-dependent gene regulation.

In a preferred embodiment, oligonucleotides or compositions of the invention are administered to an organism for the prophylaxis or treatment of a disease. The organism is preferably an animal, more preferably a mammal, and most preferably a human known or suspected to be at risk for or suffering from a disease. Diseases amenable to treatment with the inventive silencing compounds include those in which gene expression is disrupted, e.g., one or more genes are overexpressed or expressed at inappropriate times or in response to inappropriate stimuli. In preferred embodiments, oligonucleotides or compositions of the invention are administered to an organism for the prophylaxis or treatment of cancer. The invention has been exemplified herein for liver cancer.

As described in greater detail below, the silencing compound is introduced, by any convenient technique, into a cell capable of methylating oligonucleotide sequences. Most cells are capable of polynucleotide methylation and therefore the invention can be employed therein. Exemplary cells include those discussed above. In preferred embodiments, oligonucleotides are introduced into eukaryotic, plant, or prokaryotic cells, with mammalian and human cells being particularly preferred.

Oligonucleotides of the invention can be introduced into a cell in vivo or ex vivo. A variety of approaches for introducing oligonucleotides into cells in vivo and ex vivo are known and can be employed in the invention. Preferred methods include lipid or liposome-based delivery (WO 96/18372; WO 93/24640; Mannino and Gould-Fogerite (1988) BioTechniques 6(7):682-691; Rose U.S. Pat. No. 5,279,833; WO 91/06309; and Felgner et al. (1987) Proc. Natl. Acad. Sci. USA 84: 7413-7417).

Oligonucleotides or compositions of the invention can be administered directly to an organism for introduction into cells in vivo. The considerations for administering the oligonucleotides and compositions of the invention are essentially the same as the considerations for administering antisense, triplex, and other oligonucleotide compositions which are capable of modulating expression of genes implicated in viral, fungal and metabolic diseases.

For prophylactic or therapeutic applications, the dose administered to an individual, in the context of the present invention, should be sufficient to effect a beneficial response in the individual over time (i.e., an effective amount). This amount, which will be apparent to the skilled artisan, depends on the species, age, and weight of the individual; the type of disease to be treated; in some cases the sex of the individual; and other factors which are routinely taken into consideration when treating individuals at risk for, or having, a disease. A beneficial effect is assessed by measuring the effect of the compound on the disease state in the individual. For example, if the disease to be treated is cancer, the therapeutic effect can be assessed by measuring the growth rate or the size of the tumor as shown below in Example 6; by measuring the production of compounds, such as cytokines, that indicate progression or regression of the tumor; and by mortality.

Dosing is dependent on the severity and responsiveness of the disease state to be treated or prevented, with the course of treatment lasting until a beneficial effect is achieved or, in the case of prophylaxis, for as long as required to prevent onset of the disease. Optimal dosing schedules can be calculated from measurements of drug accumulation in the body of the individual. Persons of ordinary skill can readily determine optimum dosages, dosing methodologies, and repetition rates. Optimum dosages may vary depending on the relative potency of individual oligonucleotides and can generally be estimated based on effective concentration in 50% of test subjects (EC50) found to be effective in in vitro and in vivo animal models. In general, suitable doses range from 0.01 μg to 100 g per kg of body weight, and may be given once or more daily, weekly, monthly or yearly, or even once every 2 to 20 years. Persons of ordinary skill in the art can readily estimate repetition rates for dosing based on measured residence times and concentrations of the administered oligonucleotide in bodily fluids or tissues.

Following successful treatment, it may be desirable to have the individual undergo maintenance therapy to prevent the recurrence of the disease, wherein the oligonucleotide is administered in maintenance doses, ranging in dosage from 0.01 μg to 100 g per kg of body weight, and ranging in frequency from once or more daily, to once every 20 years.

### EXAMPLES

### Example 1

The following materials and methods are common to all of the examples. Test oligonucleotides are in 200 μM solutions. Cells are inoculated into 6-well microtiter plates in a 1000 μL volume, in numbers ranging from 1-5×10⁵ cells/well depending on the doubling time of the cell line. The target is to have 30-50% confluency by 24 hours after plating and before transfecting. Microtiter plates are incubated under standard conditions of 37° C., 5% CO₂, 95% air and 100% relative humidity. Oligofectamine transfection reagent (Invitrogen, Carlsbad, Calif.) is prepared by diluting one part Oligofectamine reagent with nine parts RPMI-1640 (which cannot contain serum or antibiotics) and incubating at room temperature for 5-10 min. To prepare the test compounds, 5 μL of test solution is diluted by mixing with 175 μL of serum-free, antibiotic-free RPMI-1640 or other media. Then 180 μL of diluted test solution is added to 20 μl of diluted Oligofectamine, mixed gently and allowed to stand at room temperature for 15-20 min. Next the cells are washed once with serum-free, antibiotic-free RPMI or other media by adding 800 μL solution to each well. Then the 200 μL of test solution is added to each well. The plate is gently shaken to mix the solutions. The cells are incubated for 4 hrs at 37° C. Then each well receives 1000 μL RPMI with twice the normal concentrations of serum and antibiotics. Care should be taken to avoid disturbing the transfection mixture. The mixture is incubated for 48-96 hrs, after which the cells are collected for endpoint analysis (mRNA or protein).

An oligonucleotide having the sequence: 5′-AGCCm5CGGGCTGGGAGGAGTm5CGG-3′ (“Hep22M”; SEQ ID NO: zz) was designed to target the most proximal promoter of Igf2 (human hP4 and mouse mP3) and have the five 5′ nucleotides (second strand) hybridize to the next five nucleotides (first strand) to form a hairpin. A truncated oligonucleotide (“Hep19M”) lacks three of the 5′ nucleotides and thus cannot form a loop; it has the sequence: 5′-Cm5CGGGCTGGGAGGAGTm5CGG-3′ (SEQ ID NO: xx). Hep22M and Hep19M were synthesized as phosphorothioate deoxyoligonucleotides using standard automated phosphoramidite chemistry and were purified by HPLC. The Hep19M oligonucleotide had the same sequence as Hep22M, but has three fewer bases at the 5′-end of Hep22M and thus fails to form the hairpin duplex.

More specifically, Hep22M was synthesized using phosphorothioate deoxynucleotide precursors, except that a methylated cytidine precursor (5mdC) was used to introduce methylated cytidines at desired positions in the oligonucleotide. Hep22M was synthesized as a single stranded oligonucleotide and, after HPLC purification, was dissolved in aqueous solution. In this solution, the methylator segment of Hep22M (5′-AGCCm5C-GGGCT-3′ SEQ ID NO: gg) self-anneals to form a semi-methylated hairpin structure without a linker.

This example demonstrates that Hep22M that has the semi-methylated hairpin methylator segment can inhibit Igf2 expression in liver cancer Hep 3B cells, while the truncated Hep19M that lacks the methylator segment cannot inhibit Igf2 expression.

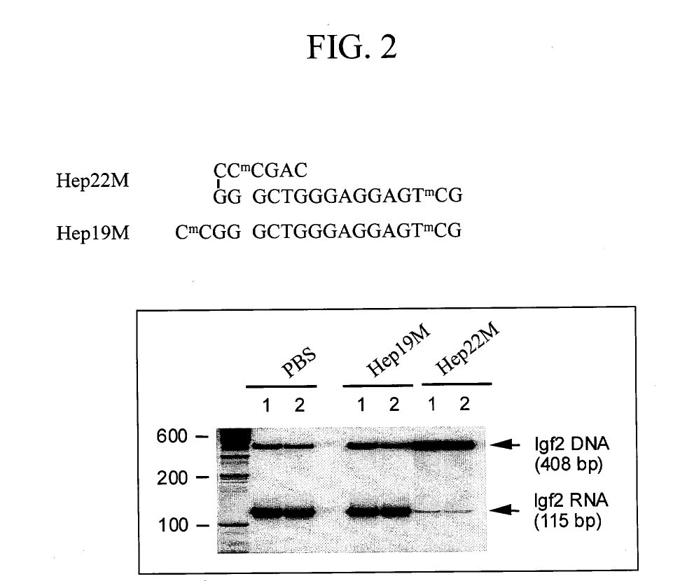

Hep 3B cells were seeded in 6-well plates and were treated with Hep22M (2 μM), Hep19M oligonucleotide (2 μM), or phosphate buffered saline (PBS) for 48 hours; and total nucleic acid was extracted and reverse transcribed to produce cDNA. Igf2 expression was quantitated by polymerase chain reaction (PCR) using a primer set that simultaneously amplified genomic Igf2 DNA and cDNA. FIG. 2 shows that the administration of Hep22M with a full methylator segment inhibited almost all Igf2 expression in Hep 3B cells, and that Hep19M that lacks the methylator segment failed to inhibit Igf2 expression.

### Example 2

This example demonstrates the anti-tumor activity of Hep22M in nude mice. Under anesthesia, nude mice (n=10-13, four weeks old, 20-25 g) were transplanted with human liver cancer Hep 3B cells in the upper left lobe of the liver. Four weeks after tumor transplantation, animals were randomly divided into four treatment groups, receiving Hep22M (10 mg/kg) (n=13), Hep19M (10 mg/kg) (n=13), cisplatin (3 mg/kg) (n=10), or PBS (n=10) through the tail vein, twice per week for as long as they survived or to a maximum of 20 weeks. The Mantel-Cox log rank test shows a significantly prolonged survival in animals receiving Hep22M, compared to those receiving Hep19M (p<0.05).

Data demonstrated that Hep22M that contains the methylator segment inhibited Igf2 expression and protected animals from tumor death. Hep19M that lacks the methylator segment cannot inhibit Igf2 expression and does not protect against tumor death.

### Example 3

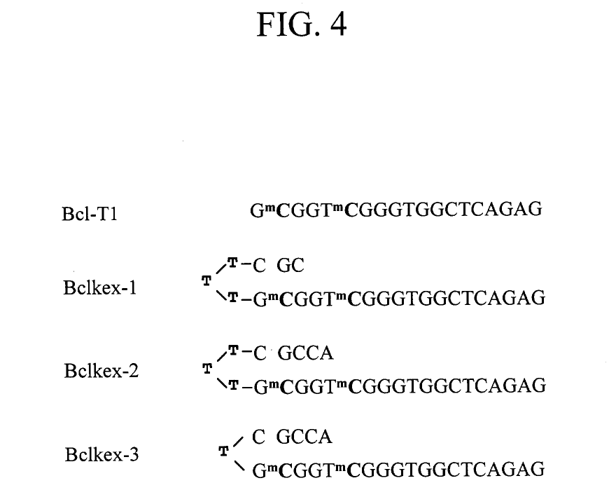

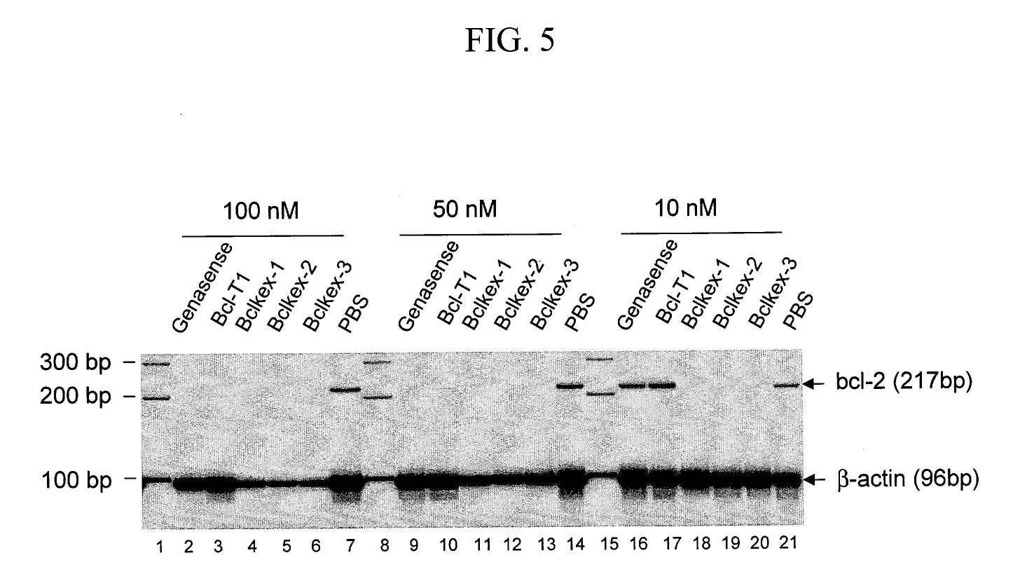

This example also demonstrates that presence of a methylator element significantly enhances the inhibition of the bcl-2 oncogene. Silencing compounds were encapsulated in liposomes (GenePORTER™ Transfection Reagent; Gene Therapy Systems, Inc., San Diego, Calif.) before application. FIG. 4 shows the compositions of four compounds. Three share the same 3′ DNA binding segment which is complementary to a transcription factor binding site in the bcl-2 promoter. Bcl-T1 did not contain a methylator segment; whereas, the other three contructs contained methylator segments. MCF-7 breast cancer cells were seeded in 24-well plates and treated for 48 hours with varying concentrations (10 nM, 50 nM and 100 nM) of the four constructs and Genasense™, an antisense Bcl-2 oligonucleotide (Genta, Inc., Berkeley Heights, N.J.). Oligos were encapsulated in liposomes (GTS delivery system). After treatment, total RNA was extracted and analyzed for Bcl-2 expression by PCR. Bcl-T1, which did not contain the hairpin structure of the other three compounds, did not block Bcl-2 expression at 10 nM level (FIG. 5). On the other hand, all three oligos with the semi-methylated hairpin structure showed a complete inhibition of Bcl-2 in MCF-7 cells at all three concentrations. This example demonstrates that the semi-methylated hairpin structure is required for maximal activity.

### Example 4

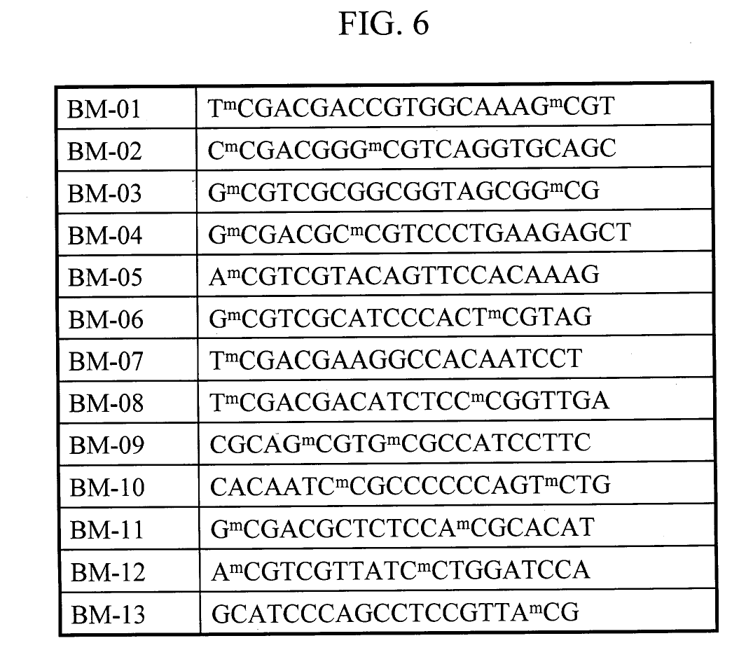

This example deals with inhibition of bcl-2 by silencing compounds containing nucleotide sequences designed to target introgenic CpG islands. The compounds have varied methylator segments depending upon the sequence of DNA binding segments (FIG. 6). The methylator segments are either linked to the 3′-end (BM-10 and BM-13) or the 5′-end (BM-01 through BM-09, BM-11 and BM-12) of the DNA binding segments. MCF-7 breast and H23 lung cancer cells were seeded in 6-well plates and were treated for 72 hrs with test compounds (1 μM) encapsulated in liposomes (GTS delivery system). After treatment with these compounds, total RNA was extracted for expression analysis of Bcl-2.

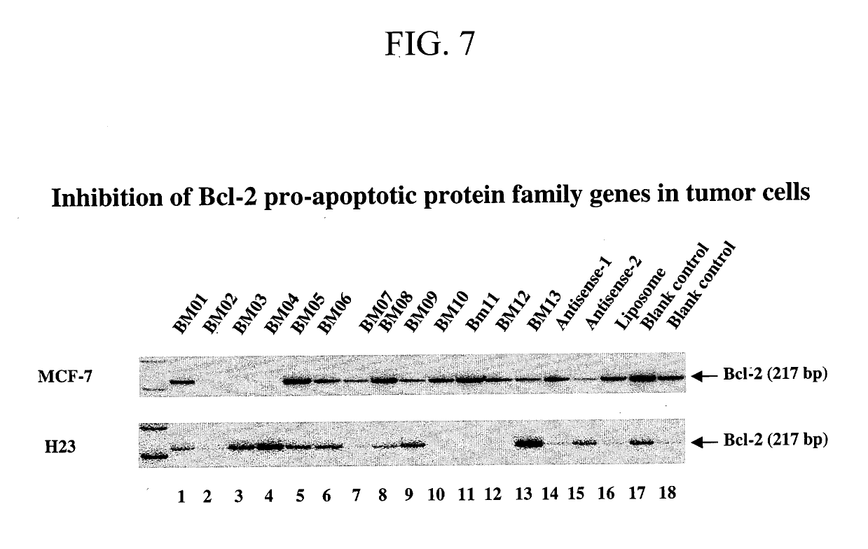

FIG. 7 shows differential inhibition of bcl-2 in two human tumor cell lines. Inhibition of Bcl-2 depended upon the sequence of the DNA binding segment of the construct. BM-02, BM-03 and BM-04 successfully inhibited Bcl-2 production in MCF-7 cells; whereas BM-10, BM-11 and BM-12 inhibited Bcl-2 production in H23 cells.

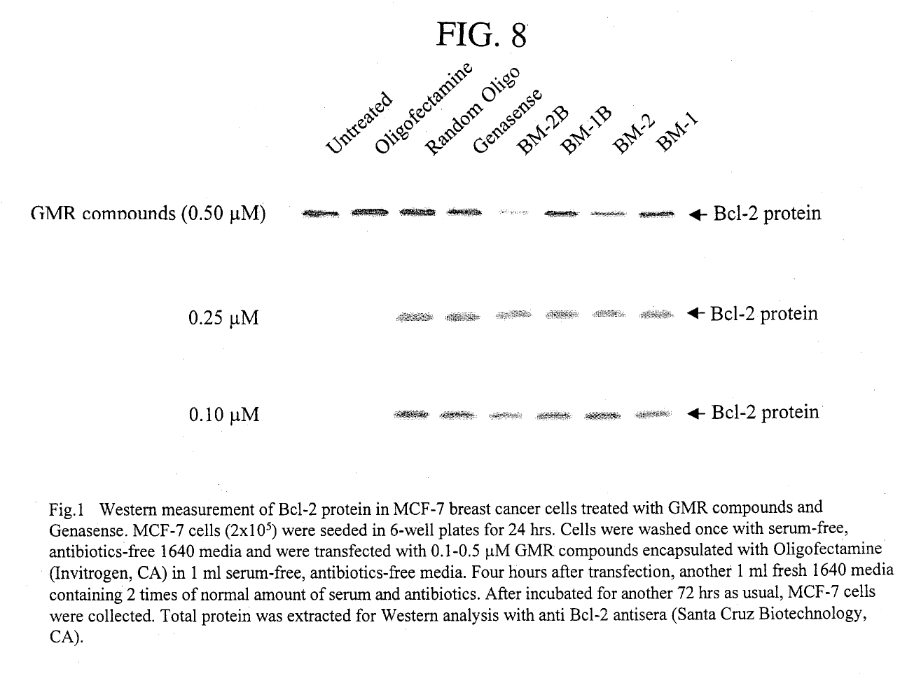

In another experiment with MCF-7 breast cancer cells, optimized compounds BM-2B and BM-1B (see Table 5, in which they are shown as Bcl2-02B and Bcl2-01B) and controls were administered and incubated for four hours. As shown in FIG. 8, BM-2B was much more effective than all other compounds.

### Example 5

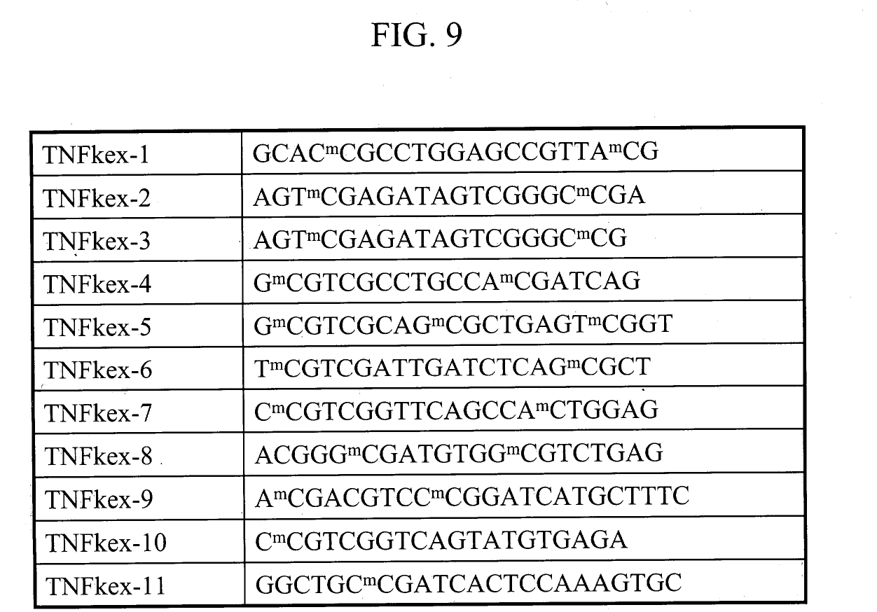

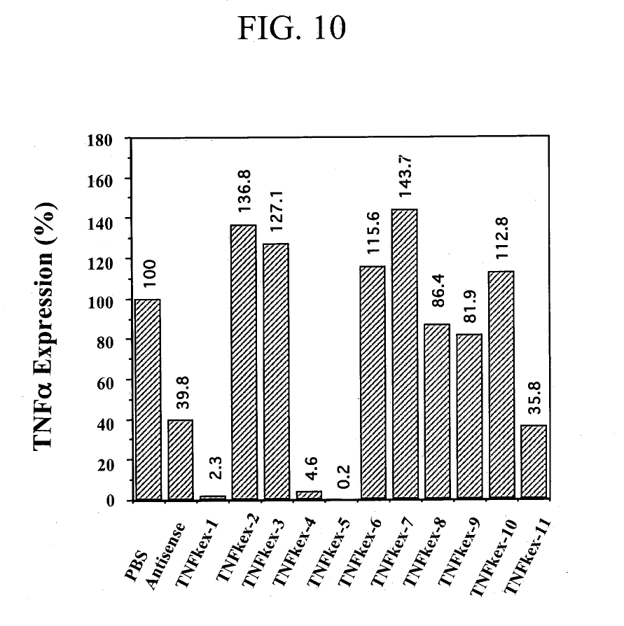

This example demonstrates inhibition of tumor necrosis factor alpha (TNFα) by silencing compounds. FIG. 9 shows the sequences of silencing compounds containing semi-methylated duplexes at either the 5′-end (TNFKex-5, -6, -7, -9, -10, -11) or the 3′-end (TNFKex-1, -2, -3, -4, -8). FIG. 10 shows the effects on TNFα production in the T47D lung cancer cell line. T47D cells were seeded in 24-well plates and treated with test compounds (0.25 μM) for 48 hours. Before treatment, the test compounds were encapsulated in liposomes using the GenePorter™Transfection Reagent (GTS) delivery system (Gene Therapy System, San Diego, Calif.). Total RNA was extracted and analyzed for TNFα expression by PCR. TNFα expression was first normalized against an internal control (β-actin mRNA) and then expressed as relative inhibition over a PBS control (100%). The results showed that the test compounds inhibited TNFα expression to varying degrees. Among them, three silencing compounds showed the most profound inhibition of TNFα: TNFKex-1, TNFKex-4 and TNFKex-5.

### Example 6

Eleven modified phosphorothioate oligonucleotides targeting a promoter sequence in the 5′-end of the MKP-1 gene (see Table 3) and eight constructs (see Table 2) targeting the CDC25A gene were prepared by SynGen Inc. (San Carlos, Calif.). All were 21-22 nucleotides in length. Compounds were synthesized using a modified ABI 392 DNA synthesizer. Salts and trace organic impurities were removed from crude products using the Applied BioBystems (Foster City, Calif.) oligonucleotide purification cartridge (OPC™) system. No further purification was carried out in this round of compound testing and optimization. Test compounds were dissolved in Tris buffer (10 mM Tris HCl, pH 8.5) for use. Either GenePorter I Transfection Reagent (GTS) or Oligofectamine™ transfection reagent (Invitrogen, Carlsbad, Calif.) was used for compound encapsulation.

MCF-7 and T47D human breast cancer cell lines purchased from American Type Culture Collections (Manassas, Va.) were used. Cell lines were maintained in RPMI-1640 media supplemented with 10% fetal bovine serum, 100 U/ml penicillin and 100 mg/ml streptomycin. Cells were seeded in 6-well plates at densities of 4×10⁵ cells/ml (MCF-7) and 5×10⁵ cells/ml (T47D). Twenty-four hours after plating, the cells were treated with 1 μM liposome-encapsulated test compounds prepared in 1 ml of fresh media, or with buffer and liposome carriers alone. After 24 hours of such treatment, 1 mL of fresh media containing serum and antibiotics was added to each well; and the cells were allowed to grow for 48 hours until they became confluent, whereupon they were harvested for RNA and protein analyses. Based on initial screening outcomes (data not shown), three constructs targeting MKP-1 and three constructs targeting CDC25A were selected for further assessment.

For mRNA expression analysis by real-time PCR, first total cellular RNA was extracted using TRI Reagent RNA isolation reagent (Sigma, St. Louis, Mo.) following procedures recommended by the manufacturer. To eliminate DNA contamination, samples were first treated with DNAse I. cDNA was synthesized with RNA reverse transcriptase as described by Hu et al. (Mol Endocrinol 1995 9:628; J Bio Chem 1996 271:28153). In a typical reaction mixture, aliquots of 2.0 μL RNA (200 μg/mL) under a 12 μL evaporation barrier of liquid was (Chill-Out™ Liquid Wax, MJ Research, Inc., Waltham, Mass.) were treated with 1.0 μL of 0.4 U DNAse I (Stratagene, La Jolla, Calif.) in 25 mM Tris (pH 8.0), 25 mM NaCl, 5 mM MgCl₂, and 0.15 U RNAse inhibitor (5′-3′, Boulder, Colo.) at 37° C. for 15 min, followed by enzyme denaturation at 75° C. for 10 min. After DNA digestion, RNAs were reverse-transcribed into cDNAs with murine leukemia reverse transcriptase (Gibco BRL, Gaithersburg, Md.) in the presence of random hexamers at 37° C. for 45 min followed by five cycles of 50° C. for 20 sec and 37° C. for 5 min (Hu et al., Mol Endocrinol 1995 9:628; Vu et al. J Bio Chem 1996 271:9014).

Transcription of MKP-1 and CDC25A mRNA was measured in MCF-7 cells by real-time PCR using an ABI Prism 7900HT Sequence Detection (Taqman™) System (ABI). PCR amplification was performed on 3.0 μL reaction mixtures, each consisting of 1.5 μL of 2×SYBR Green PCR Master Mix containing 0.25 μM of each primer and 1.5 μL of cDNA. Initial denaturation at 95° C. for 10 minutes was followed by 45 cycles of denaturation at 95° C. for 15 sec and annealing/extension at 62° C. for 1 min.

Data were collected and analyzed using software provided by the manufacturer. The abundances of CDC25A and MKP-1 mRNA were inferred from cycle threshold (Ct) values, as Ct is inversely proportional to the log of the initial template amount (copy number) and is lowest in a given data set for reactions where the initial copy number is highest.

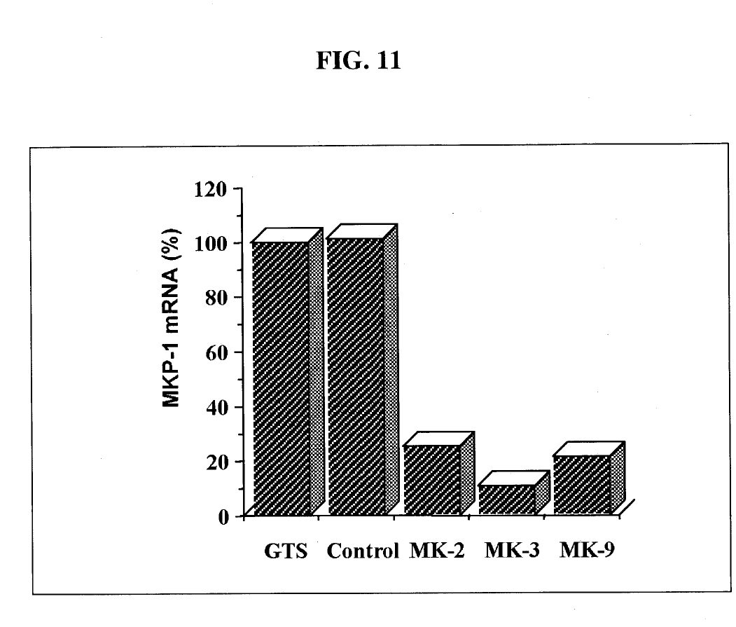

Real-time PCR was used to measure MKP-1 mRNA in MCF-7 tumor cells treated with MK-2, MK-3 and MK-9. The L-7 gene was used as an internal control for RT-PCR quantitation. As shown in Table 6, there were no dramatic differences in Ct values for L-7. Mean Ct values for MKP-1 were in the range of 31-32 cycles for GTS (liposome carrier only) and blank control groups. Mean Cts for MKP-1 increased to 35-36 cycles for groups treated with MK compounds indicating marked reductions of MKP-1 mRNA. FIG. 11 is a bar graph showing GTS and blank control groups at 100% mRNA and MK-2, MK-3 and MK-9 at low levels of mRNA.

TABLE 6
MKP-1 mRNA quantitation: Ct values

| | GTS | Control | MK2 | MK3 | MK9 |
|---|---|---|---|---|---|
| L-7 | 26.27 | 25.26 | 26.53 | 25.78 | 27.01 |
| | 26.33 | 24.95 | 26.79 | 27.23 | 26.62 |
| | 26.35 | 25.02 | 27.01 | 25.93 | 26.38 |
| Mean | 26.32 | 25.08 | 26.78 | 26.31 | 26.67 |
| MKP-1 | 32.33 | 31.58 | 35.58 | 37.36 | 35.24 |
| | 33.19 | 31.01 | 34.67 | 35.07 | 35.45 |
| | 32.41 | 31.58 | 35.01 | 35.14 | 35.06 |
| Mean | 32.64 | 31.39 | 35.09 | 35.86 | 35.25 |
| Delta-Ct¹ | 6.33 | 6.31 | 8.31 | 9.54 | 8.58 |
| dd-Ct² | 0.00 | −0.02 | 1.98 | 3.21 | 2.25 |
| MKP-1 (%)³ | 100.00 | 101.00 | 25.00 | 11.00 | 21.00 |

¹Delta-Ct = Ct (MKP-1) − Ct (L-7) (equivalent to MKP-1/L-7 ratio)
²dd-Ct = Delta Ct (treated group) − Delta Ct (GTS control)
³Copy numbers calculated by the formula 2 (dd-Ct) and then converted into % of MKP-1 mRNA

For measurement of MKP-1 and CDC25A proteins, Western blots were prepared. First, cells treated as described above were washed twice with cold PBS and then lysed with lysis buffer (50 μM tris HCl, pH 8.0, 1% NP-40, 0.25% sodium deoxycholate, 150 mM NaCl, 50 μg protease inhibitor cocktail (Roche Molecular Biochemicals, Indianapolis, Ind.)). Cell lysates were lightly mixed on a rotating wheel at 4° C. for one hr, then aspirated through a hypodermic syringe assembly (26 gauge needle) ten times. Lysates were microcentrifuged at 4° C. for 25 min, and the supernatants were collected for Western blot analyses. Lysate protein concentrations were quantitated using the DC Protein Assay (Bio-Rad Laboratories, Hercules, Calif.). Proteins (50 μg/well) were separated by gel electrophoresis (15% polyacrylamide gel), then transferred to Hybond-ECL nitrocellulose blotting paper (Amersham Biosciences, Piscataway, N.J.). The blot was incubated with blocking solution (5% non-fat dry milk in PBS (Invitrogen) containing 0.05% Tween-20) at room temperature for 1 hr and then exposed to MKP-1 or CDC25A antisera (MKP-1: Cat #SC-370 or #1199; CDC25A: Cat #7389; Santa Cruz Biotechnology, Santa Cruz, Calif.) for one hr at room temperature. Antibodies were suspended in 1% non-fat dry milk in PBS (Invitrogen) containing 0.05% Tween-20 (PBS-T). The membrane was washed thrice for 15 min with PBS-T, then incubated at room temperature for 1 hr with anti-mouse IgG conjugated to horseradish peroxidase (1:2000 dilution) prepared in the same solution as the primary antibody. After the membrane was washed three times with PBS-T, then ECL Western blotting detection reagents (Amersham Biosciences) were used to detect MKP-1 and CDC25A proteins.

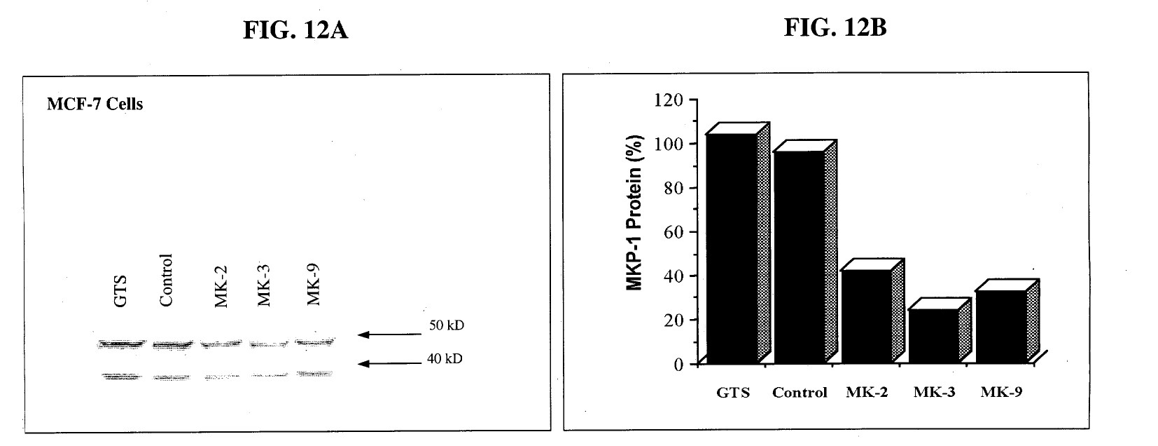

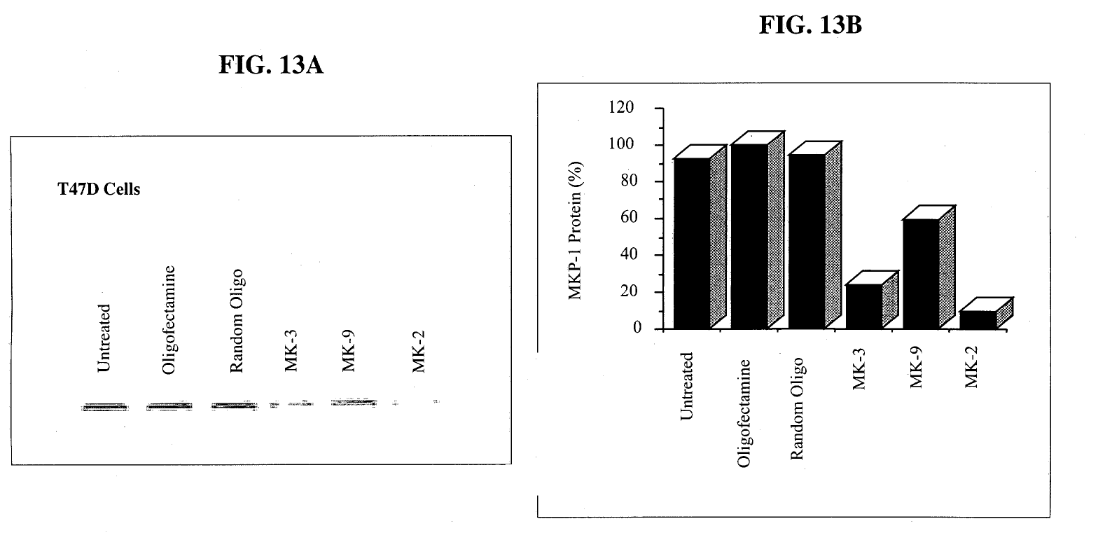

As shown in FIGS. 12A and 12B, MKP-1 protein was strongly expressed in MCF-7 cells under control conditions but was significantly reduced after treatment with compounds MKP-02, MKP-03 and MKP-09. FIG. 12A shows double bands obtained with polyclonal anti-MPK-1 antisera (Cat #1199). The intensity of the presumptive MKP-1 band was analyzed by optical densitometry and values were expressed as a percentage of the GTS control in the bar graph FIG. 12B. Similar results were obtained in T47D cells (FIGS. 13A and 13B) where a different anti-MKP-1 antisera (Cat #SC-370) yielded the expected single band. FIG. 13B shows the values expressed as a percentage of the Oligofectamine control group.

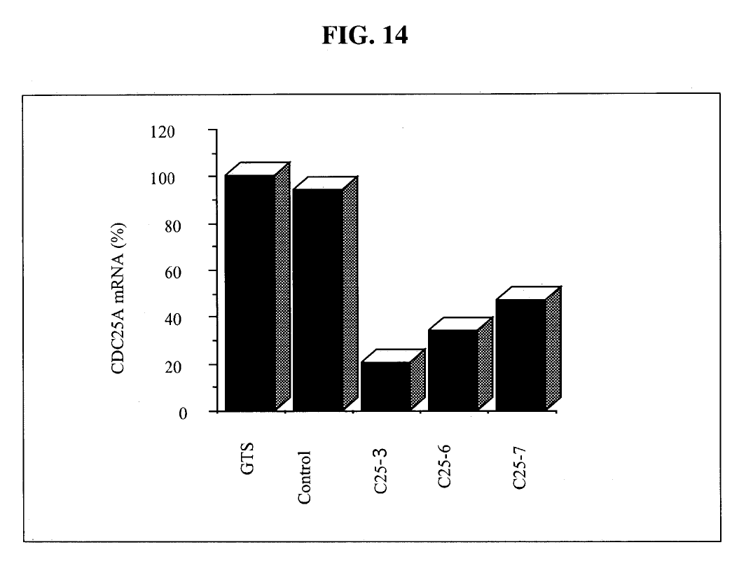

CDC25A mRNA was measured in MCF-7 cells by RT-PCR. Treatment with CDC25 compounds (CDC25-3, CDC25-6 and CDC25-7) significantly inhibited CDC25A transcription as shown in Table 7 and FIG. 14. FIG. 14 shows the percent inhibition of CDC25A mRNA as a percent of mRNA found with the GTS control.

TABLE 7
CDC25A mRNA quantitation: Ct values

| | GTS | Control | C25-3 | C25-6 | C25-7 |
|---|---|---|---|---|---|
| L-7 | 26.27 | 25.26 | 26.61 | 27.65 | 26.48 |
| | 26.33 | 24.95 | 26.80 | 27.60 | 26.00 |
| | 26.35 | 25.02 | 26.37 | 27.30 | 25.37 |
| Mean | 26.32 | 25.08 | 26.59 | 27.51 | 25.95 |
| CDC25A | 31.19 | 31.72 | 33.82 | 36.59 | 34.12 |
| | 32.45 | 31.16 | 35.35 | 35.86 | 33.46 |
| | 33.95 | 31.27 | 36.13 | 33.39 | 32.21 |
| Mean | 32.53 | 31.38 | 35.10 | 35.28 | 33.26 |
| Delta-Ct | 6.22 | 6.30 | 8.51 | 7.77 | 7.31 |
| dd-Ct | 0.00 | 0.09 | 2.30 | 1.55 | 1.10 |
| CDC25A | 100.00 | 94 | 26 | 34.1 | 46.8 |
| % | | | | | |

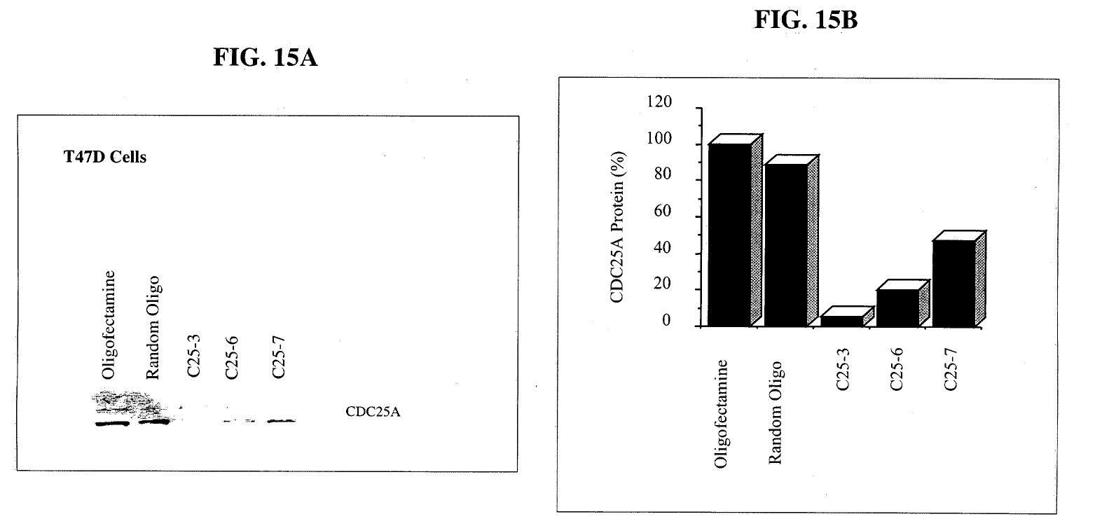

CDC25A protein expression was determined by Western blot assay of CDC25A protein in T47D breast cancer cells treated with CDC25 test compounds (FIG. 15A). Band intensity was analyzed by optical densitometry, and values were expressed as a percentage of the Oligofectamine control group (FIG. 15B).

The present invention has of necessity been discussed herein by reference to certain specific methods and materials. It is to be understood that the discussion of these specific methods and materials in no way constitutes any limitation on the scope of the present invention, which extends to any and all alternative materials and methods suitable for accomplishing the ends of the present invention.

As any person skilled in the art of designing and testing oligos for gene therapy will recognize from the previous description and from the figures and claims, modifications and changes can be made to the preferred embodiments of the invention without departing from the scope of the invention defined in the following claims.

We claim:

1. An oligonucleotide capable of silencing a target gene comprising:
a single stranded oligonucleotide DNA binding segment and an oligonucleotide methylator segment;
the DNA binding segment being complementary to either the template (non-sense) or sense strand of a nuclear DNA nucleotide sequence;
the methylator segment being double stranded and having a first strand comprising one end of the DNA binding segment and a second strand complementary to the first strand, the first or second strand comprising at least one m5CG sequence which is complementary to the target nucleotide sequence and is paired with an unmethylated CG sequence on the second or first strand so that the pairing of the first and second strands forms a semi-methylated stem loop; wherein the methylator segment and the DNA binding segment are operably linked to form a oligonucleotide capable of silencing the target gene.

2. The oligonucleotide of claim 1 wherein the first strand and the second strand of the methylator segment are linked through a covalent linkage.

3. The oligonucleotide of claim 1 wherein the single stranded DNA binding segment comprises one or more m5CG or one or more m5CN3G sequence, wherein N3 is any nucleotide.

4. The oligonucleotide of claim 2 wherein the first strand and the second strand of the methylator segment are linked by one or more nucleotide residues.

5. The oligonucleotide of claim 4 wherein the first strand and the second strand of the methylator segment are linked by one or more thymidine residues.

6. The oligonucleotide of claim 1 wherein the first strand of the methylator segment comprises Gm5CG or Gm5CN1G, and the second strand comprises CGC or GCN2G, wherein N1 is complementary to N2.

7. The oligonucleotide of claim 1 wherein the methylator segment comprises the sequence 5′-Gm5CG-T-CGC-3′ (SEQ ID NO: zz), of which the T is the linker of two strands.

8. The oligonucleotide of claim 7 wherein CGC is the first strand which is operably linked to the DNA binding segment and is complementary to the target nucleotide sequence.

9. The oligonucleotide of claim 7 wherein the Gm5CG is the first strand which is operably linked to the DNA binding segment and is complementary to the target nucleotide sequence.

10. The oligonucleotide of claim 1 wherein the methylator segment is a self-annealed double stranded duplex, forming a semi-methylated hairpin structure with no intervening linker.

11. The oligonucleotide of claim 1 wherein the DNA binding segment is about 10 to about 50 nucleotides in length.

12. The oligonucleotide of claim 10 wherein the DNA binding segment is a 22-nucleotide sense oligomer having the sequence 5′-AGCCm5CGGGCTGGGAGGAGTCGG-3′ (SEQ ID NO: zz), the 5′ AGCCm5C being the second strand and complementary to the first strand GGGCT to form a self-annealed duplex, the two strands forming a semi-methylated hairpin structure.

13. The oligonucleotide of claim 1 wherein the target nucleotide sequence is in a gene.

14. The oligonucleotide of claim 13 wherein the target nucleotide sequence is a sequence within the target gene's regulatory region, comprising promoters, exons, introns, splicing sites, 5-end or 3′-end untranslated regions, poly A signals, trans factor binding sites, enhancers, silencers, suppressors, imprinting centers, and CG islands.

15. The oligonucleotide of claim 14 wherein the target gene encodes a protein of unknown function.

16. The oligonucleotide of claim 14 wherein the target gene may be a disease gene, and the oligonucleotide can be used in gene target validation.

17. The oligonucleotide of claim 14 wherein the target nucleotide sequence is in a regulatory region of the human Igf2 gene.

18. The oligonucleotide of claim 17, wherein the target sequence is the most proximal promoter.

19. A composition comprising the oligonucleotide of claim 1 and pharmaceutical carriers and excipients.

20. The composition of claim 19 wherein the pharmaceutical carriers and excipients comprise a substance that facilitates entry of the oligonucleotide into a cell.

21. The composition of claim 20 wherein the substance comprises one or more lipids.

22. The composition of claim 21 wherein the one or more lipids comprise the cationic lipids N-[1-(2,3-dioleyloxy)propyl]-n,n,n-trimethylammonium chloride (DOTMA), dioleoyl phosphotidylethanolamine (DOPE) and/or dioleoyl phosphatidylcholine (DOPC).

23. The composition of claim 19 wherein the additional component is a physiologically acceptable carrier.

24. A method for silencing a target gene in a cell comprising introducing the oligonucleotide of claim 1 into a cell comprising the target gene, the oligonucleotide hybridizing to a nucleotide sequence of the target gene, thereby silencing the target nucleotide sequence.

25. The method of claim 24 wherein the cell is a mammalian cell, a plant cell, or a prokaryotic cell.

26. The method of claim 25 wherein the mammalian cell is a human cell.

27. The method of claim 24 wherein the oligonucleotide is introduced into the cell in vivo.

28. The method of claim 24 wherein the oligonucleotide is introduced into the cell ex vivo.

29. The method of claim 24 wherein the oligonucleotide is introduced as a composition comprising a lipid.

30. The method of claim 24 additionally comprising the step of determining a phenotypic change associated with silencing of the target gene.

31. The method of claim 30 wherein the target gene encodes a protein of unknown function.

32. The method of claim 24 additionally comprising the step of producing an organism from the cell, wherein the target sequence in a gene is silenced, whereby the organism either does not express the gene or expresses the gene at a reduced level compared to a normal organism.

33. The method of claim 24 wherein the target nucleotide sequence is in a disease gene.

34. The method of claim 33 wherein the disease gene causes cancer.

35. The oligonucleotide of claim 1, wherein the methylator segment comprises a short modified oligonucleotide, with both 3′- and 5′-ends hybridizing to each other to form a self-annealed hairpin stem with one or more optional unpaired nucleotides in the middle of the methylator segment, the unpaired nucleotide forming the loop.

36. The oligonucleotide of claim 1 wherein the methylator segment is operably linked to the 3′-end or 5′-end of the DNA binding segment.

37. The oligonucleotide of claim 1, wherein the semi-methylated stem contains more than one CG dinucleotide, wherein at least one C is a 5′-methyl cytosine (m5C) in one of the two strands, so that after self-annealing it forms a hairpin structure with one or more semi-methylated CG in the methylator stem.

38. The oligonucleotide of claim 1, wherein the stem of the methylator hairpin structure comprises two or more nucleotides.

39. The oligonucleotide of claim 1 wherein the loop of the methylator hairpin structure comprises at least one nucleotide.

40. The oligonucleotide of claim 1 wherein the methylator loop comprises at least one of T, A, G, or C, which is not complementary to another nucleotide in the methylator loop.

41. The oligonucleotide of claim 1 wherein the DNA binding segment contains one or more CG dinucleotides, whose “C” (cytosine) is a 5′-methyl cytosine (m5C).

42. The oligonucleotide of claim 1 wherein the DNA binding segment comprises at least six nucleotides.

43. The oligonucleotide of claim 1 wherein the oligonucleotide is modified by replacing two or more cytosines (C) with 5′-methyl cytosine (m5C) at two or more CG sites.

44. The oligonucleotide of claim 1 wherein the target gene sequence is a sequence in a gene promoter, enhancer, exon, intron, splicing site, 3′-untranslated region, 5′-untranslated region, or other regulatory element.

45. The oligonucleotide of claim 1 wherein the oligonucleotide comprises a deoxyribonucleic acid (DNA) backbone.

46. The oligonucleotide of claim 1 wherein the oligonucleotide comprises a ribonucleic acid (RNA) backbone.

47. The oligonucleotide of claim 1 wherein the oligonucleotide comprises a peptide nucleic acid (PNA) backbone.

48. The oligonucleotide of claim 1 wherein the oligonucleotide comprises a DNA and RNA chimeric structure.

49. The oligonucleotide of claim 1 wherein the oligonucleotide comprises a DNA and PNA chimeric structure.

50. The oligonucleotide of claim 1 wherein the oligonucleotide comprises an RNA and PNA chimeric structure.

51. The oligonucleotide of claim 1 wherein the oligonucleotide comprises a single stranded nucleotide, a portion of which forms a double stranded nucleotide by self-complementary annealing.

52. The oligonucleotide of claim 51 wherein the double stranded nucleotide is a DNA/DNA homoduplex flanked with an oligonucleotide loop comprising (N)ₙ, wherein N can be A, T, G or C, and n is at least 1.

53. The oligonucleotide of claim 51 wherein the double stranded nucleotide is a DNA/RNA heteroduplex flanked with an oligonucleotide loop comprising (N)ₙ, wherein N is A, T, U, G or C, and n is at least 1.

54. The oligonucleotide of claim 51 wherein the double stranded nucleotide is a RNA/RNA homoduplex flanked with an oligonucleotide loop comprising (N)ₙ, wherein N is U, T, G or C, and n is at least 1.

55. The oligonucleotide of claim 51 wherein the double strand nucleotide is a DNA/PNA heteroduplex flanked with an oligonucleotide loop comprising (N)ₙ, wherein N is U, A, T, G or C, and n is at least 1.

56. The oligonucleotide of claim 51 wherein the double strand nucleotide is a RNA/PNA heteroduplex flanked with an oligonucleotide loop comprising (N)ₙ, wherein N is U, A, T, G or C, and n is at least 1.

57. The oligonucleotide of claim 51 wherein at least one cytosine residue at CG sites of DNA/DNA homoduplex is replaced with 5′-methyl cytosine (m5C).

58. The oligonucleotide of claim 51 wherein at least one cytosine residue at CG sites of DNA/RNA heteroduplex is replaced with 5′-methyl cytosine (m5C).

59. The oligonucleotide of claim 54 wherein at least one cytosine residue at CG sites of RNA/RNA homoduplex is replaced with 5′-methyl cytosine (m5C).

60. The oligonucleotide of claim 55 wherein at least one cytosine residue at CG sites of DNA/PNA homoduplex is replaced with 5′-methyl cytosine (m5C).

61. The oligonucleotide of claim 56 wherein at least one cytosine residue at CG sites of RNA/PNA homoduplex is replaced by 5′-methyl cytosine (m5C).

62. The oligonucleotide of claim 59 wherein the double stranded oligonucleotide is formed through self-annealing, flanked by a short oligonucleotide loop at one side and at least one base of thymidine (T) or uracil (U) overhanging at the other side, T or U not being complementary to the target sequence.

63. A method of silencing the expression of the human Bcl-2 gene, the method comprising administering a compound selected from
Tm5CGACGACCGTGCAAAGm5CGT,
Tm5CGACGACCGTGGCAAAGm5CGTC,
Cm5CGACGGGm5CGTCAGGTGCAGC,
Cm5CGACGGGm5CGTCAGGTGCAGCT or a combination thereof.

64. A method of silencing the MKP-I gene, the method comprising administering a compound selected from
GAGm5CGGCAGTCCAGCCGCAGm5CG,
Gm5CGACGCACTGCCCAGGTACAG,
Tm5CGTCGAGCACAGCCATGGm5CGG, or a combination thereof.

65. A method of silencing the CDC25A gene, the method comprising administering a compound selected from
TGm5CGGACCCTCCAGGCGCTGm5CG,
Tm5CGACGACTCCGm5CGG7FFCAGG,
Cm5CGTCGGCAACCAGCTGTAAG,
or a combination thereof.
